(19) United States
(12) Patent Application Publication
(10) Pub. No.: US 2020/0230432 A1
(43) Pub. Date: Jul. 23, 2020

(54) TEEETH WHITENING DEVICE AND METHOD

(71) Applicant: JASIBO, LLC, Las Vegas, NV (US)

(72) Inventor: Jennifer Jablow, New York, NY (US)

(21) Appl. No.: 16/661,239

(22) Filed: Oct. 23, 2019

Related U.S. Application Data
(60) Provisional application No. 62/795,200, filed on Jan. 22, 2019.

(51) Int. Cl. A61N 5/06; A61C 19/06; A61C 17/22

(52) U.S. Cl. CPC A61N 5/0603; A61C 19/066; A61C 17/22; A61N 2005/0629; A61N 2005/0663; A61N 2005/0652; A61N 2005/0606

(57) ABSTRACT

The present disclosure relates to devices, methods and kits for accelerating the rate of whitening of teeth.

### CROSS-REFERENCE TO RELATED APPLICATIONS

This application claims priority to and the benefit of U.S. Provisional Patent Application Ser. No. 62/795,200 filed Jan. 22, 2019, the disclosure of which is incorporated herein by reference in its entirety.

### FIELD

The present disclosure includes devices and methods for accelerating the rate of whitening of teeth using an oxidizing agent, a photocatalytic agent, a pH adjuster and a portable light source.

### BACKGROUND

Whiteness of teeth can be an important cosmetic desired element. Common teeth-bleaching method can include oxidizing agents such as hydrogen peroxide, which can be used in a liquid form, and which is mixed with an anhydrous carrier containing glycerin and/or propylene glycol and/or polyethylene glycol. Another oxidizing agent is carbamide peroxide (urea hydrogen peroxide) which can be dissolved in ethanol and which upon contact with water dissociates into urea and hydrogen peroxide. Hydrogen peroxide in the presence of water dissociates into water and an oxygen free radical, the latter being highly reactive and, as a result, react with the stain molecules in teeth making them water soluble and/or transparent or both.

Another approach is to incorporate the oxidizing agent into a strip and have the patient wear the strip intermittently over a period of, for example, two weeks. This approach also has the disadvantage of being awkward for the patients to wear, and many patients wearing the strips also experience tooth sensitivity.

Professional tooth whitening can be performed in a dental office and may include the application of light activation in combination with peroxides and oxidizing agents. Home use peroxides are regulated substances that have concentrations that are generally substantially lower than those used in professional settings. The concentration of the oxidizing agent can define the rate of whitening, and therefore home treatments have suffered from significantly lower whitening rates than that available in professional dental treatments.

Tooth whitening with LED light has become popular in the last decade. Most popular has been the light in the blue wavelength spectrum. The thought process has been that blue light is a complimentary color to yellow stains in teeth and that will work most efficiently. The present disclosure is focused on the development of a home dental product which accelerates the whitening rate by using photocatalytic methods in the violet/indigo part of the electromagnetic spectrum to increase the concentrations of oxidizing agents at the time of treatment.

### SUMMARY

In one embodiment, an illuminating device is provided. The illuminating device includes an LED light source that generates a wavelength of light of from about 400 nm to about 420 nm that is coupled to a removable mouthpiece that acts as the light guide for precisely directing the light to the teeth.

In another embodiment, a method of tooth whitening is provided. The method includes preconditioning the tooth surface with one or more photocatalytic agents and one or more oxidizing agents, and irradiating the tooth surface with light of a wavelength from about 400 nm to about 420 nm from an LED light source.

An illuminating device for whitening the teeth in the mouth of a user is provided. The illuminating device includes a light source that generates a wavelength in the violet portion of the electromagnetic spectrum in a wavelength of light of from about 400 nm to about 420 nm that is coupled to a mouthpiece.

An oral treatment device for whitening the teeth in the mouth of a user is provided. The oral treatment device includes a mouthpiece and a control unit. The mouthpiece is sized and shaped so that it can be inserted into the user's oral cavity and includes an inner border having a substantially concave shape to substantially match the curvature of rows of the teeth in the mouth, a light generating element positioned to focus the light generated toward the teeth positioned in the mouthpiece and a plurality of projections on the inner border of the mouthpiece positioned adjacent the teeth positioned in the mouthpiece. The control unit is connected to the mouthpiece and includes a vibration generating device for generating vibrations to cause the mouthpiece to vibrate and a controller. The controller is configured to selectively energize the vibration generating device and selectively energize light generating element.

A method of whitening the teeth in the mouth of a user using an oral treatment device is provided. The oral treatment device includes a mouthpiece and a control unit. The mouthpiece is sized and shaped so that it can be inserted into the user's oral cavity and includes an inner border having a substantially concave shape to substantially match the curvature of rows of the teeth in the mouth, a light generating element positioned to focus the light generated toward the teeth positioned in the mouthpiece and a plurality of projections on the inner border of the mouthpiece positioned adjacent the teeth positioned in the mouthpiece. The control unit is connected to the mouthpiece and includes a vibration generating device for generating vibrations to cause the mouthpiece to vibrate; and a controller. The controller is configured to selectively energize the vibration generating device and selectively energize light generating element. The method includes applying a tooth whitening composition to the teeth; placing the teeth into the mouthpiece of the oral treatment device; applying vibrations to the teeth by energizing the vibration generating device of the oral treatment device and applying light to the teeth by energizing the light generating element of the oral treatment device.

### DESCRIPTION OF THE DRAWINGS

The accompanying drawings illustrate presently preferred embodiments of the present disclosure, and together with the general description given above and the detailed description given below, serve to explain the principles of the present disclosure.

FIGS. 1A and 1B are illustrations of an illuminating device embodiment of the present disclosure;

FIG. 2 is an illustration of a mouthpiece embodiment of the device embodiment of FIGS. 1A AND 1B;

FIG. 3 is an illustration of illuminator embodiment of the device embodiment of FIGS. 1A AND 1B;

FIG. 4 is an illustration of an illuminating device embodiment of the present disclosure of the device embodiment of FIGS. 1A AND 1B;

FIG. 5 is a rear perspective view of a device embodiment of the present disclosure; and

FIG. 6 is a top view of the device embodiment of FIG. 5;

FIG. 7 is a bottom view of the device embodiment of FIG. 5;

FIG. 8 is a first side view of the device embodiment of FIG. 5;

FIG. 9 is a side view opposite to the view in FIG. 8 of the device embodiment of FIG. 5;

FIG. 10 is a rear view of the device embodiment of FIG. 5;

FIG. 11 is a front view of the device embodiment of FIG. 5;

FIG. 12 is a top view of the device embodiment of FIG. 5 showing the inside of control unit;

FIG. 13 is an illustration of a block diagram of the electrical components of the device embodiment of FIG. 5;

FIG. 14 is an illustration of a block diagram of an exemplary computing/processor apparatus;

FIG. 15 is a top perspective view of the mouthpiece of the device embodiment of FIG. 5;

FIG. 16 is a top view of the mouthpiece of the device embodiment of FIG. 5;

FIG. 17A is a perspective view of the device embodiment of FIG. 5 showing the inside of the upper section of the control unit;

FIG. 17B is a perspective view of the device embodiment of FIG. 5 showing the inside of the lower section of the control unit;

FIG. 18 is a cross-sectional view of the mouthpiece of the device embodiment of FIG. 5;

FIG. 19 is a cross-sectional view of the mouthpiece of the device embodiment of FIG. 5 and an exemplary implementation thereof;

FIG. 20 is a top perspective of a device embodiment of the present disclosure; and

FIG. 21 is a top view of the device embodiment of FIG. 20.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Advantages and other aspects of the exemplary embodiments included herein will become apparent from the following detailed description considered in conjunction with the accompanying drawings. It is to be understood, however, that the drawings are designed solely for purposes of illustration and not as a definition of the limits of the disclosure, for which reference should be made to the appended claims. Additional aspects and advantages of the disclosure will be set forth in the description that follows, and in part will be obvious from the description, or may be learned by practice of the disclosure. Moreover, the aspects and advantages of the disclosure may be realized and obtained by means of the instrumentalities and combinations particularly pointed out in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The aspects of the present disclosure are directed to a portable light sources or devices and dental bleaching methods that overcome certain shortcomings associated with existing whitening products and methods. The light sources or devices comprise a violet/indigo light illuminator to accelerate teeth bleaching agents. The teeth are separately preconditioned with a bleaching material, a hydrating compound (with or without an oxidizing bleaching agent) and, optionally, other treatment ingredients including, for example, a catalyst, a pH adjuster, etc. Specifically, application of the methods and devices of the present disclosure result in an increased whitening rate and shorter home treatment times.

Shorter electromagnetic wavelengths in the violet/indigo bands about 400 nm to about 418 nm (preferably violet bands including about 405 nm to about 410 nm or about 405 nm) are useful in the process of bleaching teach. It is believed that the emission of violet light (about 405-410 nm) coincides with the absorption peak of pigmented molecules in the stains (e.g., yellow stains) that are formed over time. The violet light, thereby, interacting selectively and breaking up the stain molecules into smaller and colorless ones. The speed at which the violet/indigo (preferably violet) light interacts with these stain molecules increases thousands of times, thus accelerating the breakup process. If fragments of the stain molecules do not rearrange, the molecules stop absorbing the violet/indigo (preferably violet) light and the colored center of the stain molecules disappear. This process can occur at higher or lower intensity in many objects with pigmented molecules. Dental pigment molecules (such as stain molecules) are photoreceptive, and, therefore, highly reactive to light. Their molecular chains are long and with sequences of chemical bonds that dislocate electrons and are very susceptible to absorption of shorter wavelengths, such as the violet light.

An advantage of the technique using direct violet/indigo (preferably violet) light is the good aesthetic result obtained after just a few short treatment sessions, preserving the enamel and dental structures. A violet/indigo (preferably violet) photocatalytic bleaching system can also be used in light-activated bleaching procedures using different concentrations of peroxide in bleaching agents, promoting excellent results.

It is believed that violet light has more energy carried in its photons and is more absorbed on the dental surface due to its physical characteristics. These physical violet light characteristics are believed to be responsible for larger surface absorption of light and breaking the pigments (bleaching) the stained teeth. It is also believed that violet light has the characteristic of not having a demineralization effect on the teeth and when used in a fractional way with intervals of time without continuous application does not cause heating of the teeth.

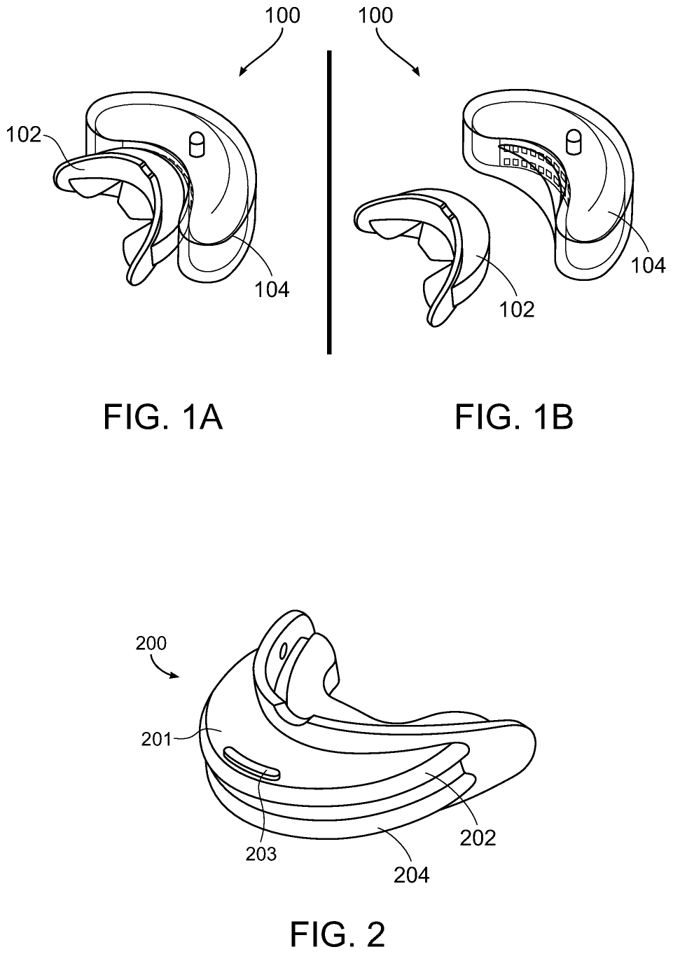

FIGS. 1A and 1B illustrate one embodiment of an illuminating device 100 comprised of a removable mouthpiece 102 that acts as the light guide for precisely directing the light to the teeth and an illuminator 104, such as, for example an LED light source, that generates the light. As used in the present disclosure, the illuminator and LED light source can include one or more light generating elements. The optically clear mouthpiece 102 snaps into the illuminator 104 during treatment as in FIG. 1A and is removed as in FIG. 1B for cleaning after each treatment. This design allows for the mouthpiece 102 to be replaced without replacing the other elements of the stem. Each mouthpiece 102 as well as other mouthpieces included in the present disclosure may also be individualized to fit precisely the dental surface. It also allows for use of multiple mouthpieces so that the device may be shared among multiple family members much the same way an electric toothbrush is shared. The detachable light source 104 can be powered by a power source as included in the present disclosure including, for example, a rechargeable battery.

FIG. 2 illustrates one embodiment of the mouthpiece illustrated in FIGS. 1A and 1B in which mouthpiece 200 includes a connector section 201 with dual cylindrical lens 202 and 204 that couples the light from a dual row of light generating elements in the light source unit (i.e., an illuminator), including for example, LEDs, precisely onto the teeth. The top and bottom rows of, for example, LEDs from the light source are focused through the upper and lower cylindrical lens 202 and 204 onto the upper and lower row of teeth respectively. Connector section 201 may include a flange 203 to aid in securing the mouthpiece to the light source to which it is attached during use. The mouthpiece is removable and consumable and is designed to facilitate light activation of whitening pre-treatments and whitening composition, for example, a catalyst without transmitting heat to the teeth.

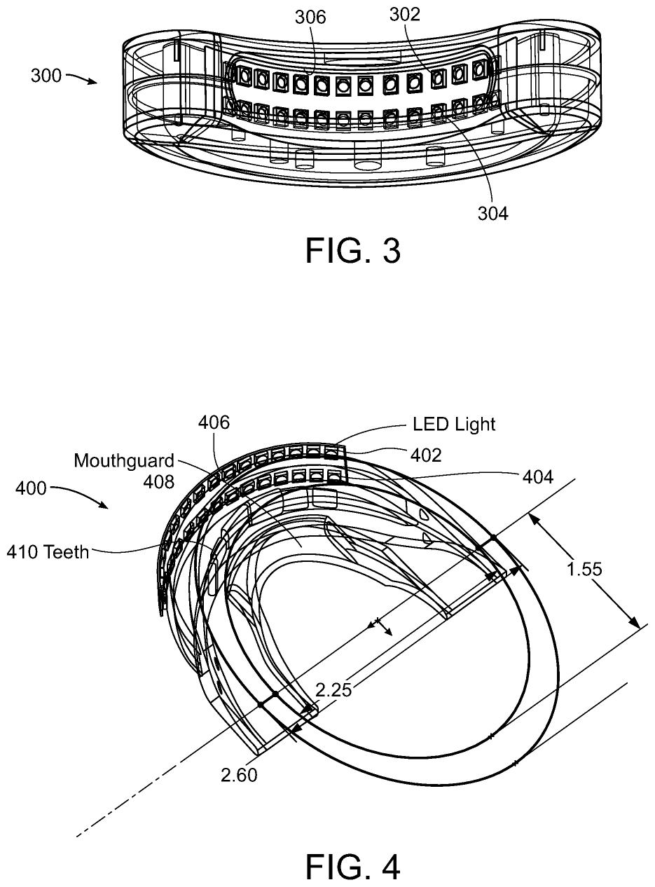

FIG. 3 illustrates one embodiment of the light source illustrated in FIGS. 1A and 1B in which the illuminator 300 itself which can be used in conjunction with mouthpiece 200 and includes a dual row light generating elements, for example, LEDs, configuration 302 and 304 which may mate with the dual cylindrical lens array 202 and 204 within the mouthpiece 200. Light source 300 also includes a receiving pocket 306 in which the connector section 201 of mouthpiece 200 is received in order to mate the dual row light generating elements, for example, LEDs, configuration 302 and 304 with the dual cylindrical lens array 202 and 204 within the mouthpiece 200, The light generating elements 302 and 304 are formed into a curved configuration to mate precisely with the curvature of the lens assemblies 202 and 204 on the front surface of the mouthpiece 200.

FIG. 4 illustrates an embodiment illustrating the novel optical design of the embodiment illustrated in FIGS. 1A, 1B, 2 and 3 including illuminating device 400, the dual light generating element arrays 402 and 404 of the illuminator 406 and the dual cylindrical lens of mouthpiece 408 which provides optimum irradiance on each row of teeth 410, and minimizes irradiance on the gums and other soft tissues.

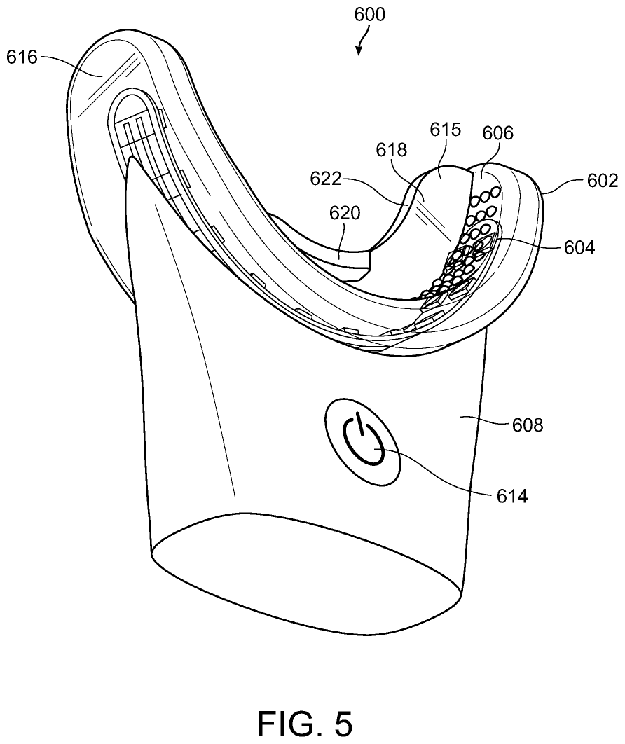

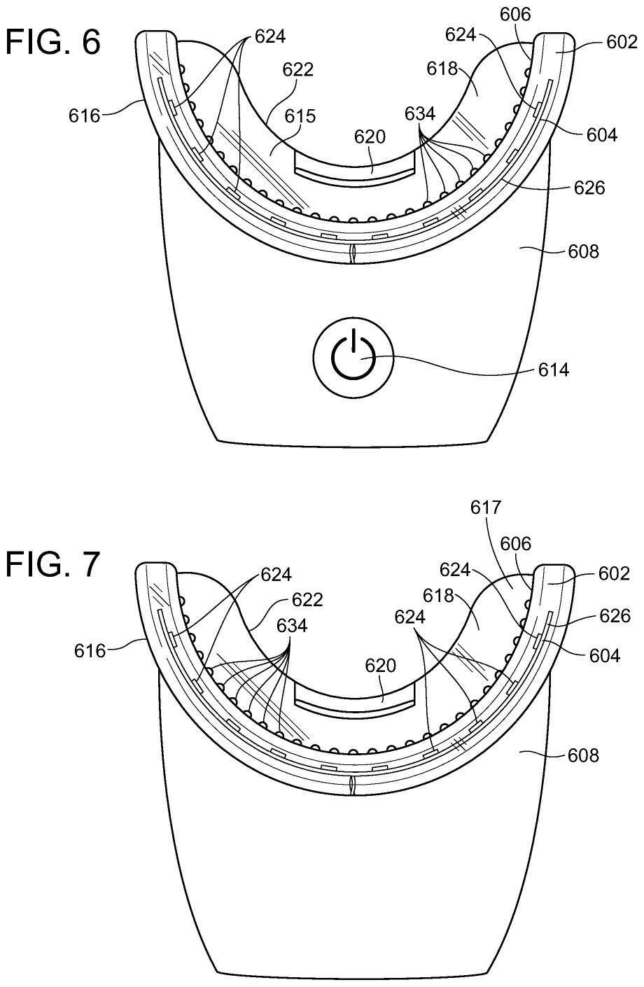

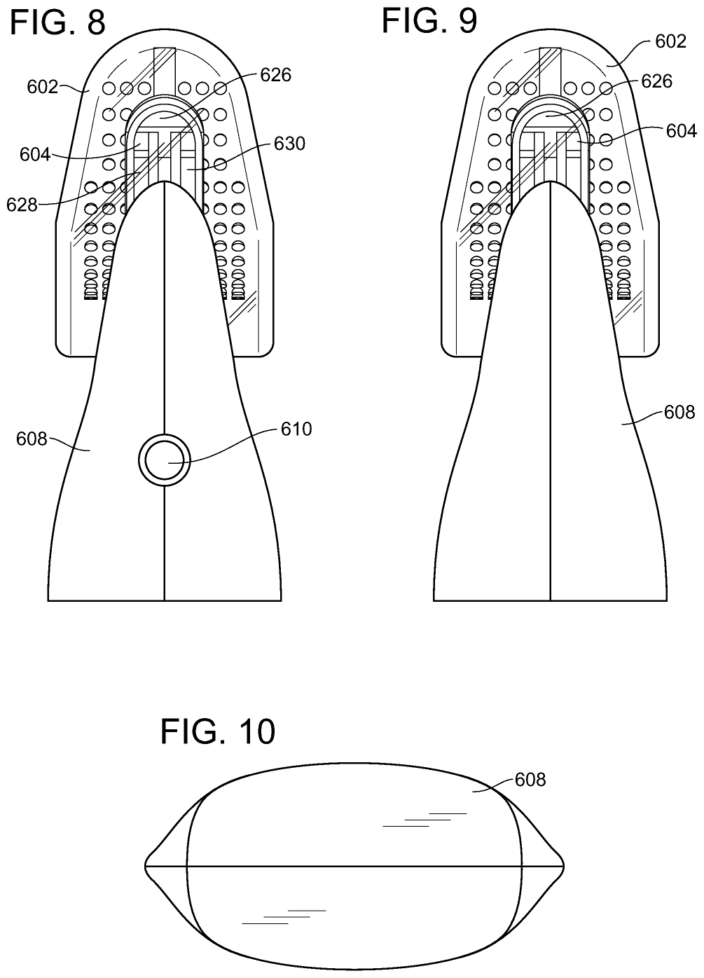

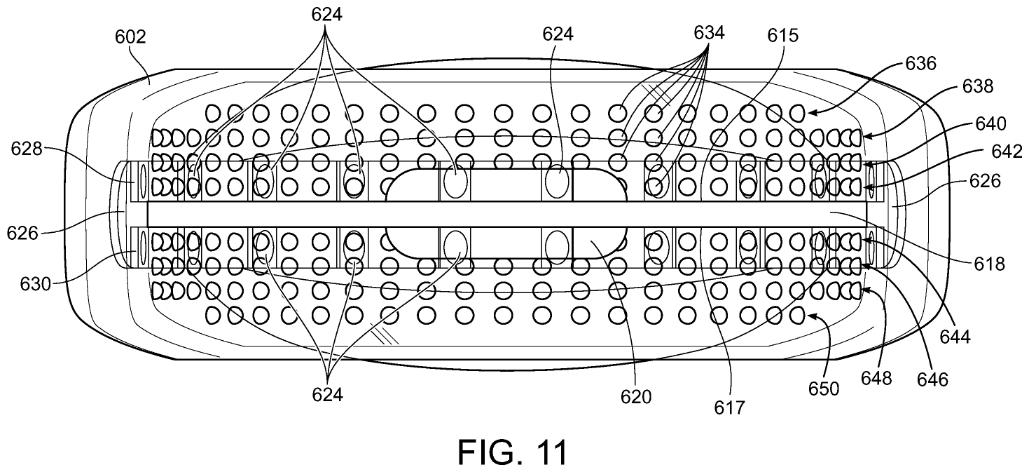

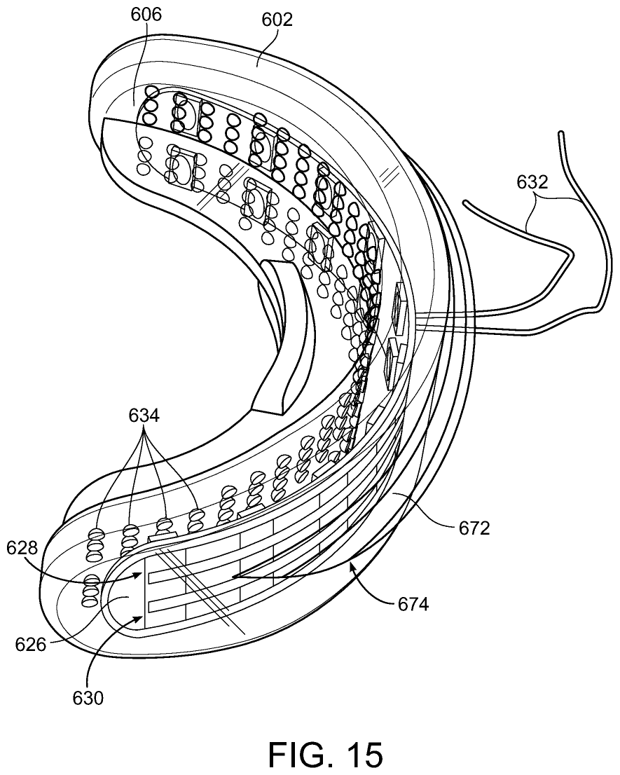

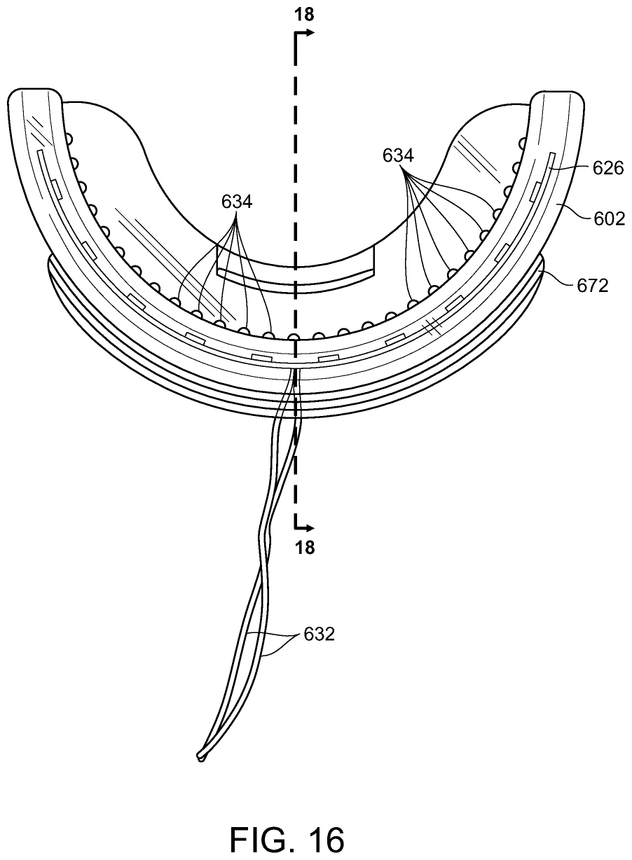

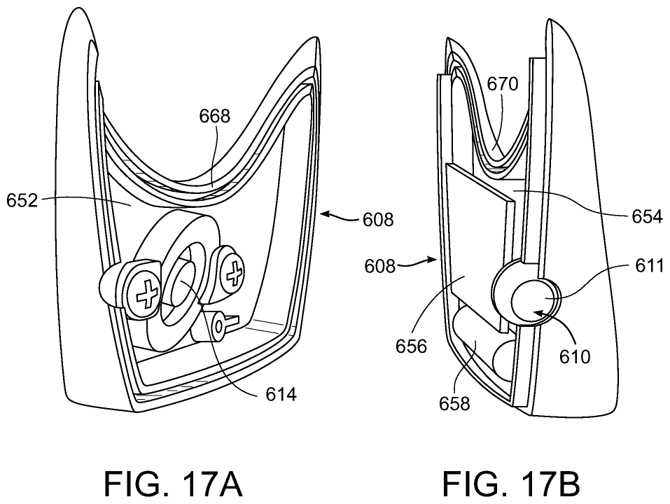

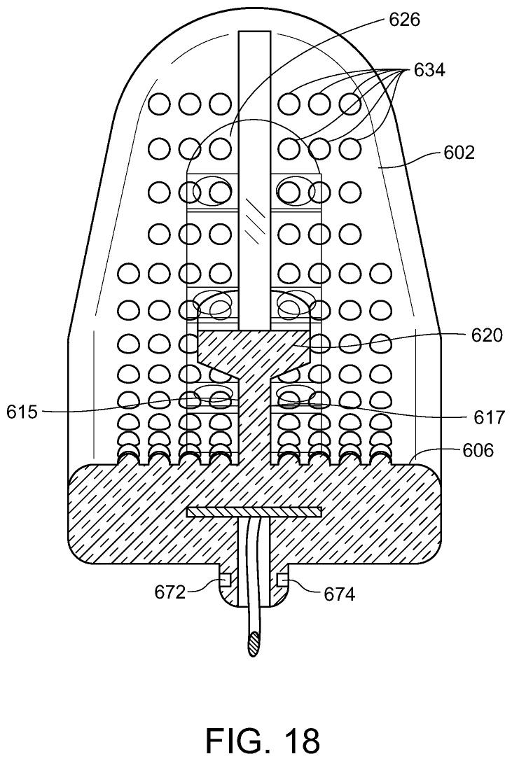

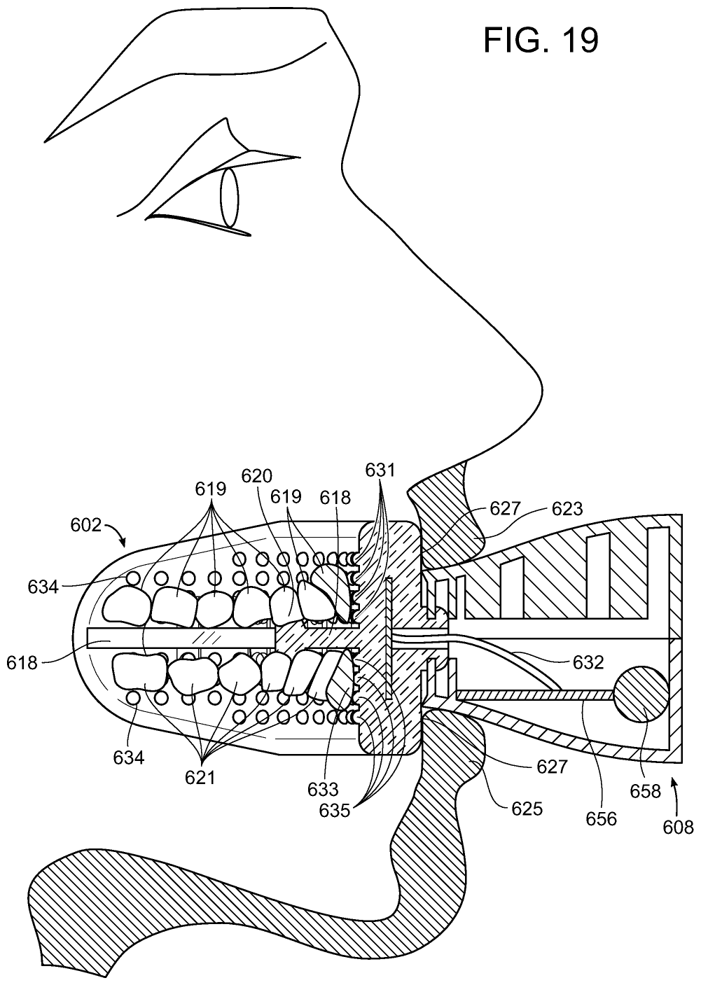

FIGS. 5-19 illustrate another embodiment of an illuminating device 600 comprised of a mouthpiece 602 which when positioned for treatment, the teeth 619 and 621 bite down on thereby placing the light generating element 604 adjacent the front tooth surface and proximal to the teeth 619 and 621 along the inner border 606 of mouthpiece 602 having a substantially concave curvature to substantially match the size and shape of the rows of teeth that are to be placed adjacent or against it and that includes a light generating element 604, such as, for example an LED light source or plurality of LED light sources, that generates the light as shown herein. The embodiment of FIGS. 5-19 include a control/power unit 608 that includes apparatus to power and control the features including the light generating element 604 as well as other features, such as, for example, apparatus to impose vibration or sonic waves on the teeth being treated and/or apparatus to pulsate, have a continuous strobe or any combination thereof of the light generation element 604 during the treatment period. The control/power unit is attached to the mouthpiece 602. The control/power unit can also include one or more charging ports that may be different connector formats, illustrated as power connector 610 and on/off button 614. In this embodiment, the lips 623 and 625 can fit over the outer boundary 627 of mouthpiece 602 as shown in FIG. 19.

Further details of the embodiment of FIGS. 5-19 include the mouthpiece is made of a material that is substantially clear or substantially transmissive of the light generated by the light generating element 604 and can be made of flexible or elastomeric that would be comfortable when engaged with the teeth and mouth of a user. The material of which the mouthpiece 602 is made should also be biocompatible and non-reactive with the pre-treatment compositions, teeth whitening compositions and components thereof included in the embodiments of the present disclosure. Such material can include, for example, silicone (including BPA free silicone) and plastic (including BPA free plastic). Mouthpiece 602 should also be sized and shaped so that it can be inserted into the user's oral cavity during use and, thus, can be considered to be intraoral in nature as shown in FIG. 19.

Mouthpiece 602 also includes a barrier plate 618 such that when the teeth of a user are positioned thereon, the upper teeth 619 can bite down thereon and the lower teeth 621 can bite up thereon with upper lip 623 and lower lip 625 comfortably positioned against the back 627 of mouthpiece 602 as illustrated in FIG. 19. The barrier plate 618 may also include a back stop 620 on both upper surface 615 and lower surface 617 of barrier plate 618 to prevent the teeth from sliding off the back edge 622 of the mouthpiece. Mouthpiece 602 also includes light generating element 604 that can include a plurality of light generating elements 624, e.g., LED lights. Light generating element 604 can be a printed circuit board (PCB) 626 that includes the plurality of light generating elements 624 disposed thereon such that the light generating elements 624 are wired such that they can be connect to wire conduits 632 that provide power to the light generating elements 624. The light generating elements 624 can be divided into two rows 628 and 630 with row 628 directed to the upper teeth and row 630 directed to the lower teeth. The number of light generating elements 624 for example in each row can be about 8-about 15, about 10. The light generating element 604 is formed into a curved configuration to approximately mate with the curvature of inner border 606 of mouthpiece 602 and the light generating elements 624 are positioned to focus their light on the teeth (e.g., including the front of the teeth) positioned in the mouthpiece 602.

The light generating elements 624 can be sufficient in number and sufficiently spaced so as to provide sufficient light irradiation toward the front of teeth, preferably including the front of all the teeth, positioned in mouthpiece 602 to bring about the whitening of the front of the teeth which have been treated with one or more composition embodiments of the present disclosure, including at a wavelength of from about 400 nm to about 520 nm or the violet portion of the electromagnetic spectrum for photocatalysis of from about 400 nm to about 420 nm (preferably about 405 to about 410 nm, preferably about 405 nm).

The inner border 606 of mouthpiece 602 can also include a plurality of projections 634 extending radially away from mouthpiece 602 and toward teeth positioned therein as described herein. The plurality of projections 634 are intended to be positioned adjacent and in contact the teeth for at least a portion of the treatment period as exemplary shown in FIG. 19 where upper front tooth 629 is positioned against projections 631 and lower front tooth 633 is positioned against projections 635 (the number of projections depending on the vertical height of the teeth in contact with inner border 606), preferably in contact during the vibration portion of the treatment period, more preferably the entire treatment period, when the teeth are positioned in mouthpiece 602. The projections are shown in an exemplary shape to be substantially round in shape and substantially rounded or semicircular in cross-section, however, other shapes can be used equally as well including, for example, but not limited to elliptical, square, rectangular, triangular, polygonal (e.g., pentagon, hexagon, etc.) The plurality of projections 634 can be sufficient in number and size outwardly extending toward the teeth positioned in the mouthpiece 602 and sufficiently spaced so as to provide at least one projection positioned adjacent and in contact each tooth positioned in the mouthpiece 602 for at least a portion of the treatment period. An exemplary number and arrange of the plurality of projections 634 is shown in FIG. 11 that includes 4 upper rows 636, 638, 640 and 642 and 4 lower rows 644, 646, 648 and 650, the group of upper rows and group of lower rows separated by barrier plate 618. Nineteen is exemplary number of projections for rows 636 and 650 that are positioned farthest from barrier plate 618 and 27 is exemplary number of projections for other rows.

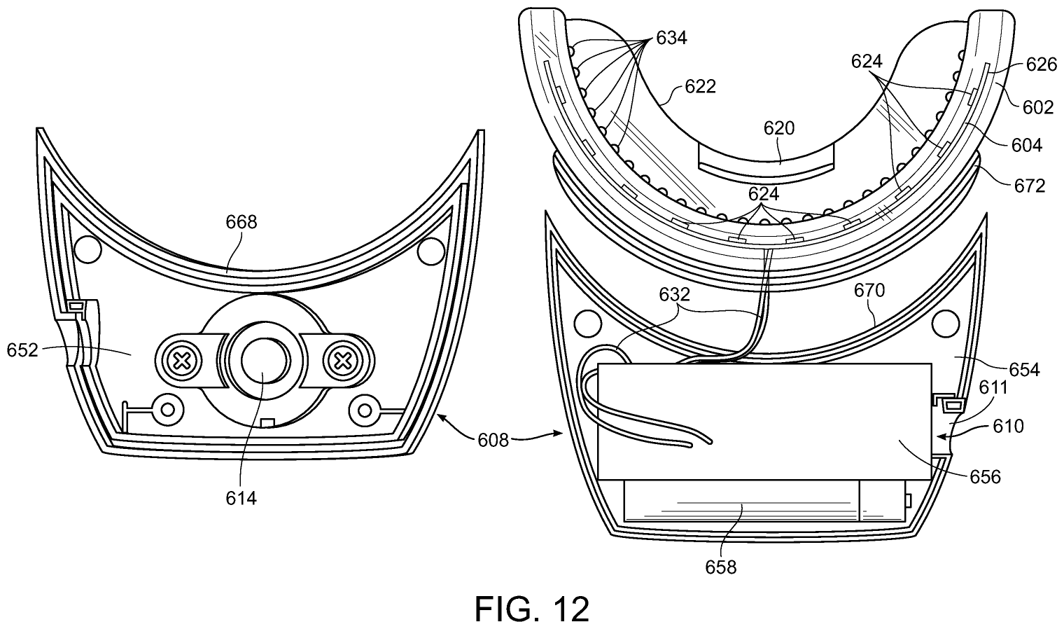

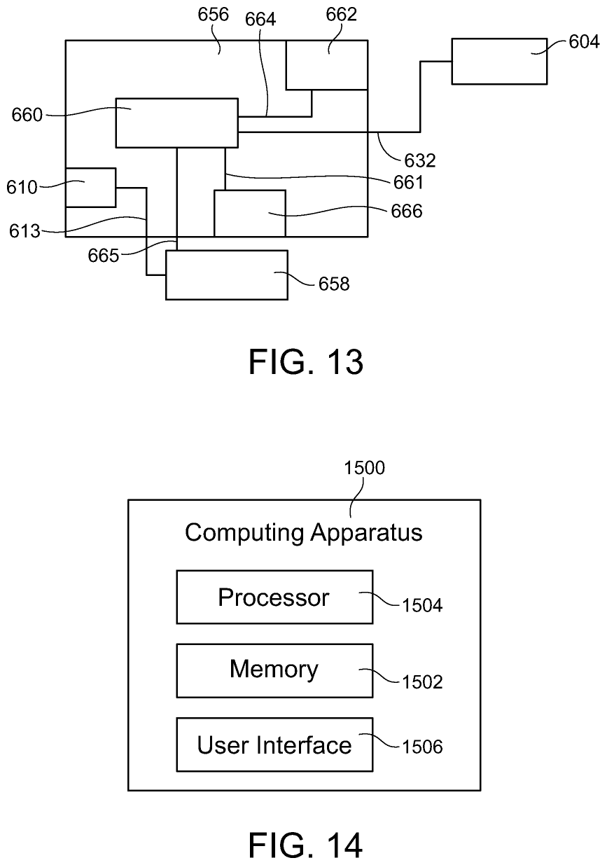

FIGS. 12 and 13 includes a view of exemplary components of the control/power unit 608. Control/power unit 608 can include an upper section 652 and a lower section 654. Upper section 652 can include on/off button 614. Lower section 654 can include a PCB 656 that is connected to wire conduits 632 that are part of mouthpiece 602 and to a power source 658 (e.g., a battery including a rechargeable battery) electrically connected to PCB 656. However, the power source need not be local to the control/power unit 608 and can be remote thereto including, for example, a remote power source via, for example, power connector 610 accessible through connector port 611 that can be connected to for example, a remote battery including a rechargeable battery, an electrical outlet, a cellphone, a computer, etc. though the use of a variety of electrical conduits and connectors (two-prong plug, three-prong plug, USB, etc.). Connector port 610 can also be used to recharge power source 658 where it is a rechargeable battery.

As the exemplary block diagram in FIG. 13 shows the PCB 656 can include a central processing unit (CPU)/controller 660, power connector 610 and a vibration generating device 662 which can be, for example, a micro-vibration motor which has an offset impeller to make the whole illuminating device 600 or at least the mouthpiece 602 vibrate for reasons explained below. CPU/controller 660 can be used to selectively energize and control the operation of the light generating element 604 and the vibration generating device 662 which includes turning on, monitoring the time period for which each is active and turning off each after a period of time for which it is programed. Vibration generating device 662 is connected via electrical conduit 664 and can also be remote from PCB 656, but local to the control/power unit 608 as long as it is capable of making the whole illuminating device 600 or at least the mouthpiece 602 vibrate. The CPU/controller 660 is connected via electrical conduit 632 to the light generating element 604. CPU/controller 660 can be connected via electrical conduit 665 to power source 658 or to a relay that is connected to power source 658 that in turn is also connected to and thereby controls the flow of power and operation of the light generating element 604 via wire conduits 632 to which it is connected and vibration generating device 662 to which it is connected via electrical conduit 664. Power connector 610 can be connected to power source 658 via electrical conduit 613. When the control/power unit 608 is assembled, on/off button 614 is positioned adjacent to so as to interact with switch 666 on PCB 656 which is also connected to CPU/controller 660 connected via electrical conduit 661 so that the user of the device can commence the whitening process performed by the illuminating device 600. Switch 666 can also be remote from PCB 656, but local to the control/power unit 608 as long as it is connected to CPU 660/controller.

In at least one aspect of the disclosed embodiments, the systems and methods disclosed herein may be executed by a controller (e.g., CPU/controller 660) including one or more computers or processor-based components under the control of one or more programs stored on computer readable medium, such as a non-transitory computer readable medium. FIG. 14 shows a block diagram of an exemplary computing apparatus 1500 that may be used to practice aspects of the disclosed embodiment. In at least one exemplary aspect, the digital receiver/processor and other disclosed devices, components and systems may be implemented using an instance or replica of the computing apparatus 1500 or may be combined or distributed among any number of instances or replicas of computing apparatus 1500.

A computing apparatus 1500 may include computer readable program code or machine readable executable instructions (such as, for example, instructions to pump fluids and operate the agitator motors utilized in the embodiments disclosed herein) stored on at least one computer readable medium 1502, which when executed, are configured to carry out and execute the processes and methods described herein, including all or part of the embodiments of the present disclosure. The computer readable medium 1502 may be a memory of the computing apparatus 1500. In alternate aspects, the computer readable program code may be stored in a memory external to, or remote from, the apparatus 1500. The memory 1502 may include magnetic media, semiconductor media, optical media, or any media which may be readable and executable by a computer. Computing apparatus 1500 may also include a processor 1504 for executing the computer readable program code stored on the at least one computer readable medium 1502. In at least one aspect, computing apparatus 1500 may include one or more input or output devices to allow communication among the components of and those connected to the system model, including, for example, operation of the light generating element 604 and vibration generating device 662 using what may be generally referred to as a user interface 1506, which may be connected to or operate as switch 666, which may operate the other components included in or connected to CPU/controller 660 or to provide input to the computing apparatus 1500 to or from other components of the system model.

Mouthpiece 602 can be firmly mounted to the control unit 608 using standard adhesives. Such mounting can also include use of a retaining rib 668 on the upper section 652 that is curved in shape and retaining rib 670 on lower section 654 that is similarly curved in shape. Such retaining ribs 668 and 670 fit into slots 672 and 674 of mouthpiece 602 that are similarly curved in shape to each other and to ribs 668 and 670 so that they when mouthpiece 602 can held in place when upper section 652 and lower section 654 of the control unit 608 are assembled.

The teeth whitening process using the embodiment of the present disclosure and included in FIGS. 5-19 commences with the user applying to his or her teeth one of the hydrating agents (e.g., mineral oil) compositions of the present disclosure or one of the desired teeth whitening compositions of the present disclosure that include an oxidizing agent and optionally including separate application of or combination with the oxidizing agent other additional compositions of the present disclosure that aid, catalyze or improve the operation of the oxidizing agent (e.g., photocatalytic agents, pH adjusters, etc.) as well as other optional ingredients (flavor, sweeteners) included herein. After application, the teeth are then positioned in and engage the embodiment of the present disclosure with and included in FIGS. 5-19, preferably so the front of the teeth are pressed against the inner border 606 of mouthpiece 602 and the plurality of projections 634 and the on/off button 614 is pressed by the user resulting in the switch 666 being activated and the treatment period begins. The CPU/controller 660 then executes instructions included therein to control operation of the vibration generating device 662 and the light generation element 604 and associated light generating elements 624.

The treatment period can commence with applying micro vibrations to the teeth after application of the compositions of the present disclosure. The micro vibrations are generated by the vibration generating device 662 that is activated and controlled by the CPU/controller 660. The micro vibrations can extend for a period of time ranging from about 10 seconds to about 60 seconds, about 15 seconds to about 30 seconds, about 20 seconds at a frequency ranging from about 300 Hz to about 350 Hz, about 325 Hz. After the above period of time for the micro vibrations, CPU/controller 660 turns off the vibration generating device 662. The micro vibrations cause the different components and compositions of the present disclosure that have been applied to the teeth to mix and better whiten the teeth to which they have been applied as a result thereof. The micro vibrations may also cause the compositions of the present disclosure that have been applied to the teeth to better permeate the surface of the teeth themselves.

The treatment period also includes the CPU/controller 660 activating the light generation element 604 and associated light generating elements 624. Such activation of the light generation element 604 can take place once the on/off button 614 is pressed by the user and the micro vibrations begin, at any time during the period of the micro vibrations are applied or after the micro vibrations have been applied, preferably about when the micro vibrations begin. The treatment period including micro vibrations and activation of the light generation element 604 to project light therefrom toward the teeth can extend for a period of time ranging from about 10 minutes to about 30 minutes, about 15 minutes to about 25 minutes, about 20 minutes. The light from the light generation element 604 can also optionally pulsate as disclosed herein. After the above period of time for the micro vibrations, CPU/controller 660 turns off the light generation element 604 once the treatment period is finished.

When the treatment period is over, the user removes their teeth from the device and wipes the teeth with a tissue or towel. The user should not preferably eat, drink or brush teeth for at least 20 minutes after treatment. The entire treatment is safe to do up to about 2 consecutive treatments for up to about 3 to 5 days in a row on heavily stained teeth to get the level of white you desire. For heavily stained teeth, it is safe to do up to about 2 consecutive about 20 minute treatments per day for about 3 days to about 5 days get your desired level of white. It is safe to whiten teeth as often as every about 30 days. Touch up treatments depend on lifestyle. Wine, smoking and certain foods stain teeth and may require more frequent treatments for about 4 or 5 days with treatments twice a day.

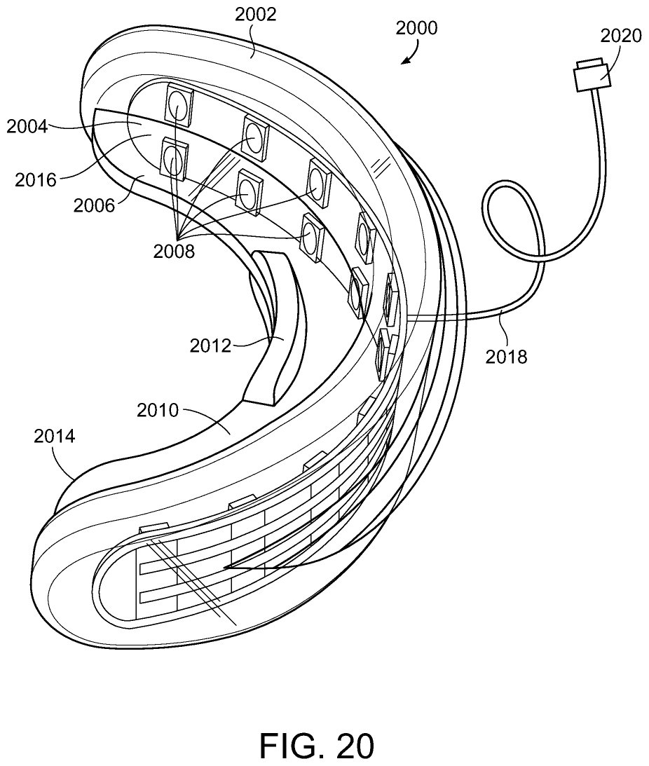

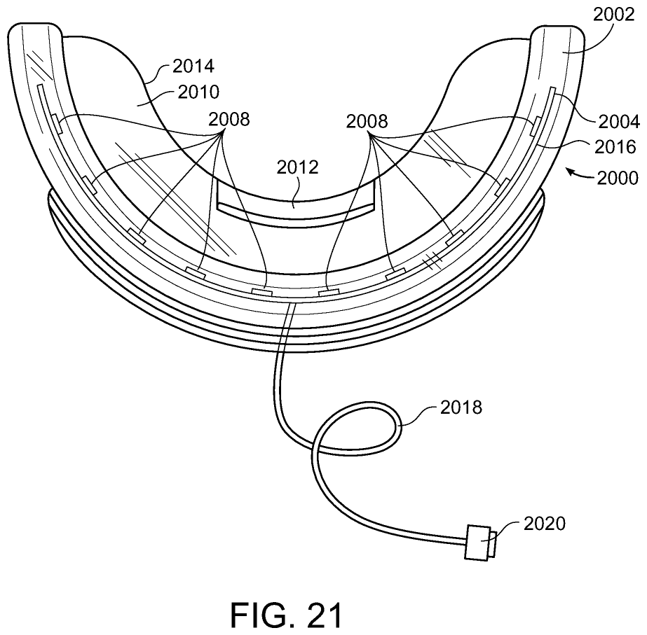

FIGS. 20 and 21 illustrate another embodiment of an illuminating device 2000 comprised of a mouthpiece 2002 which when positioned for treatment, teeth biting down thereon similar to what is shown on FIG. 19 for the previous embodiment including placing the light generating element 2004 adjacent the front tooth surface and proximal to the teeth along the inner border 2006 of mouthpiece 2002 that includes a light generating element 2004, such as, for example an LED light source or plurality of LED light sources 2008, that generates the light. Mouthpiece 2002 also includes a barrier plate 2010 such that when the teeth of a user are positioned thereon, the upper teeth can bite down thereon and the lower teeth can bite up thereon as illustrated in FIG. 19 for the previous embodiment. The barrier plate 2010 may also include a back stop 2012 to prevent the teeth from sliding off the back edge 2014 of the mouthpiece. Mouthpiece 2002 also includes light generating element 2004 that can include a plurality of light generating elements 2008, e.g., LED lights. Light generating element 2004 can be a printed circuit board (PCB) 2016 that includes the plurality of light generating elements 2008 disposed thereon such that the light generating elements 2008 are wired such that they can be connect to an electrical conduit 2018, for example, a cable or other form of electrical wiring that can provide power to the light generating element 2004.

The light generating elements 2008 can be sufficient in number and sufficiently spaced so as to provide sufficient light irradiation to the front of teeth, including the front of all the teeth, positioned in mouthpiece 2002 to bring about the whitening of the front of the teeth which have been treated with one or more composition embodiments of the present disclosure, including at a wavelength of from about 400 nm to about 520 nm or the violet portion of the electromagnetic spectrum for photocatalysis of from about 400 nm to about 420 nm (preferably about 405 to about 410nm, preferably about 405 nm). Electrical conduit 2018 can be used to provide power from an external source to the light generating elements 2008 using a connector 2020 suitable for connecting the cable to an external power source such as an electrical outlet, a cellphone, an electronic tablet, a computer, etc. though the use of a variety of connectors (two-prong plug, three-prong plug, USB, etc.).

Another embodiment of the present disclosure may include an illuminating device in which the light generating element (e.g., LED) are positioned inside the mouth while focusing the light on the teeth in order to, for example, bring the light generating element closer to the teeth. Other embodiments of the illuminating device of the present disclosure may also include apparatus to impose vibration or sonic waves on the teeth being treated. The illuminator of embodiments of the present disclosure can include light generating element numbering as many as 28 and as little as 6 but preferably 16 to 20, light generating units.

The illuminating devices of the present disclosure may all include an illuminator LED light source that generates a desired intensity, frequency and wavelength of light that can also include a removable mouthpiece similar to the embodiments illustrated in FIGS. 1A, 1B, 2, 3 and 4 as well as optionally acting as the light guide for precisely directing the light to the teeth.

The light-emitting diode (LED) array of the embodiments of the present disclosure can be housed in a waterproof or water-resistant enclosure which has an optically transmitting faceplate in front of the LED array. The portable light source can be completely self-contained and includes the LEDs, the rechargeable battery or batteries, all control and recharging electronics, and a heat management system.

The design uses light energy used in embodiments of the present disclosure of from about 400 nm to about 520 nm or in the violet portion of the electromagnetic spectrum for photocatalysis of from about 400 nm to about 420 nm (preferably about 405 to about 410nm, preferably about 405 nm). The device can use a light generating element, for example, LED lights, that are continuous, pulsate, have a continuous strobe or any combination thereof during the treatment period.

Such LED lights used in embodiments of the present disclosure typically have substrate based on zinc selenide (ZnSe), indium gallium nitride (InGaN), silicon carbide (SiC) and silicon (Si). The LED lights may comprise encapsulated clear or colored molded shells. The shells are designed to boost the light emission from the semiconductor core by acting as a diffusing lens, allowing light to be emitted at a much higher angle of incidence from the light cone than the bare chip is able to emit alone. Light size is designed for maximum tooth illumination. Lights may be of varied size depending on tooth dimensions (molar, incisor, genetically small or large, damaged teeth or gums, etc.). Tooth widths are typically 2-9 mm and thus the LED lights may optionally be of variable size so as to maximally irradiate the tooth surface. The lights are also arrayed along a cylindrical axis in which the emitted light is directed perpendicular to the tooth surface at a distance to maximize the incident intensity. Aspects of the present disclosure may also allow the user to perform mobile hands-free treatments with no connecting cords or secondary elements. The average lux output of the light generating element of embodiments of the present disclosure (e.g., PCB with LEDs) and used with the pre-treatment composition embodiments, teeth whitening composition embodiments, and related use embodiments thereof of the present disclosure should be at least 1300, greater than 1500, about 1590.

Violet/indigo (preferably violet) illumination used in embodiments of the present disclosure of the catalyst conditioned teeth may lead to so-called recombination of the catalytic dissociation products. To limit such recombination, reagents may be added and/or the light may be turned off to spatially control the localized production of ions. As such, the light may be made to strobe or pulsate (turn on and off repeatedly) to reduce the amount of recombination (the combining of substrate charge centers and/or the photocatalytic created reactive species—both of which reduce activity due to recombination). In a preferred embodiment the flashing rate is about 33 Hertz with a 5% duty cycle (5% ON time), however the strobe rate can range from about 1 Hz to about 10 kHz, about 1 HZ to about 50 HZ and the duty cycle can range from about 1% to about 75%. These ranges can be adjusted to maximize the whitening rate by increasing the short-term irradiance levels required to drive more instantaneous catalytic events, while at the same time spatially and temporally minimizing recombination events.

Embodiments of the present disclosure may also include a dual cylindrical lens array coupled with a curved dual LED array as illustrated in the embodiment of FIG. 2. This design combination maximizes the activity at and around the front of the tooth surface, including the cracks and spaces between the teeth. This design can provide superior light distribution to the anterior teeth. It can also allow for maximum activity at the tooth surface, with minimal activity on the gums and other soft tissue. The lens material may comprise optically clear polyurethane or any optically clear material that transmits blue light including silicone, PMMA (acrylic), polycarbonate, etc.

Embodiments of the present disclosure also encompasses a heat management system that stores the heat within the light source or mouthpiece in which light generating element (e.g., LED) are positioned so that it is not conducted or radiated to the teeth. Preferably the heat around the teeth is within 1-5 F. degrees of ambient oral temperature. Normal tooth temperature is about 35 to about 37° C. (95 to 98.6° F.). The thermal reservoir is sufficient in size that it can store the entire heat generated during a treatment regimen.

Embodiment of the present disclosure also allows the user to perform a rapid whitening operation in a completely remote and hands-free manner, not requiring a sink or to be connected in any way to any type of cable, tether, control device, power storage pack, or power management device.

Aspects of the present disclosure include embodiments including various compositions used for teeth whitening including pretreatment compositions and tooth whitening compositions as well as associate methods that can include the use of device embodiments of the present disclosure and methods of using same. Pre-treatment compositions of the present disclosure include compositions and components thereof that are applied prior to teeth whitening or whitener compositions. Teeth whitening or whitener compositions (also referred to as tooth whitening or whitener, bleaching or oxidizing agents) include a bleaching or oxidizing agent and optionally other compositions and components of pre-treatment compositions.

The increased whitening rate and efficiency of the present disclosure also includes methods and composition for improved photocatalytic activity of one or more photocatalytic agents used in conjunction with a tooth whitening composition such as an oxidizing agent that is applied to the tooth surface before exposure to irradiation. In this embodiment, a photocatalytic agent can be a metal salt catalyst and an oxidizing agent are used. The oxidizing or bleaching agent can be, for example, carbamide peroxide, hydrogen peroxide, calcium peroxide, zinc peroxide, sodium chlorite, sodium perborate as is hydrogen peroxide (H₂O₂). Metal salt catalysts, preferably dentally or pharmaceutically acceptable metal salt catalysts, that may be employed include, for example, magnesium gluconate and magnesium oxide, titanium dioxide, zinc oxide, zinc gluconate, iron oxide, ferrous gluconate, ferric chloride (with ferric chloride, not enough to cause an exothermic reaction) or a combination thereof, preferably zinc oxide (ZnO). The photocatalytic agent may be added to the tooth whitening oxidizing agent before being applied to the teeth or it can be applied to the teeth prior to application of the tooth whitening agent where the two component mix during the treatment process. In a preferred embodiment the photocatalytic agent (e.g., ZnO) is added to the tooth surface first, and the tooth whitening oxidizing agent (e.g., H₂O₂) is applied over the top of the photocatalytic agent (e.g., ZnO) film. The photocatalytic agent (e.g., ZnO) concentration can be between about 0.1 to about 25% by weight. In this manner the catalysis is localized specifically to a thin layer at the surface of the tooth for maximum whitening benefit. This method concentrates the catalytic dissociation of the tooth whitening oxidizing agent (e.g., H2O2) precisely at the tooth surface and further ensures that the bulk of the tooth whitening oxidizing agent (e.g., H2O2) remains optically transparent so that the light may penetrate to the surface of the tooth for maximum catalytic activity. These embodiments can be combined with the device embodiments of the present disclosure and the use thereof, such that the device is used after the photocatalytic agent and oxidizing agent are applied to the teeth.

Because these UV and other wavelengths are not desirable for soft tissue exposure, violet/indigo (preferably violet) wavelengths are employed. Also, the catalytic conversion efficiency can be dramatically decreased at longer wavelengths. A variety of absorption shifting agents may be used to enhance the efficacy of these longer wavelengths to violet/indigo (preferably violet) wavelengths. In a preferred embodiment fluorescein can be used because of its existing FDA approval status, but any number or combination of additional absorption shifting elements may be employed.

The catalysis process takes place on the surface of the photocatalytic substrate. In a preferred embodiment this substrate is a small particle of photocatalytic agent (e.g., ZnO). In a preferred embodiment these particles are applied in a thin film using a dried atomized powder. However, this film may be applied employed using either hydrophobic, hydrophilic, or amphipathic carriers. The efficiency of a singular conversion is governed by the photon wavelength, and is maximum in the violet/indigo (preferably violet) range. The rate of conversion is governed by the number of photons and the number of substrate sites. Therefore both the irradiance and photocatalytic agent (e.g., ZnO) concentrations are increased to significantly higher levels than is normally employed. The present disclosure utilizes an irradiance of between 3.5 to about 20 mW/cm2, more preferably between 5 to about 20 mW/cm2, more preferably 10 to about 15 mW/cm2. One embodiment of the disclosure utilizes an irradiance of about 11-12 mW/cm2. Another embodiment utilizes an irradiance of greater than 5 mW/cm2. photocatalytic agent (e.g., ZnO) substrate concentrations of between 0.1 to 25% by weight are common. More preferably the substrate concentrations (i.e., the concentration of the photocatalyst) are between 4 to about 10%, more preferably about 2 to about 6%, most preferably about 3%. In another embodiment, the substrate concentration is greater than about 10% by weight. In general, the specifications for irradiation and substrate concentration may range from about 0.1 to about 50 mW/cm2 and about 0.1 to about 100% respectively.

In addition to wavelength and irradiance, there are additional rate limiting factors, the first of which is the size of the photocatalytic agent substrate particle. A particle size that is similar to the wavelength of light to be absorbed can be more optimal. Another embodiment of the disclosure relates to a method of whitening wherein said photocatalytic agent (e.g., zinc oxide) is a powder USP in a gel formulation wherein the average photocatalytic agent (e.g., zinc oxide) particle size is between 0.1 microns to 100 microns, more preferably 0.5 microns to 100 microns, most preferred 10 microns. In another embodiment the particle size is approximately 400-420 nm, preferably about 405 nm to about 410 nm, about 405 nm. This can help increase absorption efficiency, but also helps spatially separate catalytic dissociation to help minimize recombination.

Another embodiment of the disclosure relates to a method of whitening kit wherein said composition of zinc oxide and said composition of hydrogen peroxide are delivered from a single component with 2 separate chambers that mix upon delivery to the tooth surface. The delivery apparatus for the zinc or peroxide includes syringe, ampoule, or tube. Such apparatus may be opaque so as to prevent damage during storage. These embodiments can be combined with the device embodiments of the present disclosure and the use thereof, such that the device is used after the above components are applied to the teeth as disclosed herein. The zinc oxide side can also have a pH adjuster preferably above 8 to help increase rate.

Another embodiment of the disclosure relates to the use of a whitening or bleaching agent (also referred to as a tooth whitening or whitener agent or oxidizing agent) alone or in combination with other methods or treatment compositions of the present disclosure. Such oxidizing or bleaching agent can include carbamide peroxide, hydrogen peroxide, calcium peroxide, zinc peroxide, sodium chlorite and sodium perborate but preferably hydrogen peroxide. The oxidizing agent, for example, hydrogen peroxide, may be formulated as a gel, dentifrice, paste, thin film, strip, liquid, spray, or paint applied to the tooth labial surface. In relation to another embodiment of the present disclosure, when the oxidizing agent is used in combination with a photocatalytic agent (e.g., ZnO), it creates an admixture with said photocatalytic agent (e.g., zinc oxide) at the tooth interface. Embodiments of the disclosure relate to a method of whitening wherein the concentration of hydrogen peroxide ranges from about 0.1 to about 35 percent by weight, more preferably about 9 to about 14 percent by weight. For a carbamide peroxide, calcium peroxide or urea peroxide base teeth whitener composition, each or a mixture can be employed at concentrations of from about 9 weight % to about 48 weight %, preferably from about 27 weight % to about 42 weight %.The present disclosure may comprise a variety of different oxidizing agents, peroxide types, and concentrations. The preferred embodiment is about 9% H₂O₂. These embodiments including the oxidizing agent alone or in combination with other methods or treatment compositions of the present disclosure can be combined with the device embodiments of the present disclosure and the use thereof, such that the device is used after the oxidizing agent is applied to the teeth.

The oxidizing agent may also include the addition of other ionic impurities (singularly or in combination) to boost the generation of active oxidizing agents. These may include sodium chloride, potassium chloride, sodium hydroxide, and a host other such agents known to those skilled in the art, including any mixture thereof.

An oral or pharmaceutically acceptable oxidizing or bleaching agent and peroxide stabilizer can be optionally used with any of the embodiments of the present disclosure and can include one or a mixture of such agents, such as, for example, ethylenediamineteraacetic acid, disodium salt; ethylenediamineteraacetic acid, tetrasodium salt; ethylenediaminetetraacetic acid, calcium disodium salt; etidronic acid; citric acid; gluconic acid; sodium citrate; sodium gluconate; sodium phosphate; disodium phosphate; trisodium phosphate; tetrapotassium pyrophosphate; sodium tripolyphosphate as well as sodium stannate and potassium stannate (tin can be a or peroxide stabilizer). The peroxide stabilizer content the oxidizing agent containing composition embodiments of the present disclosure can be in the range of from about of 0.01 weight % to about 2 weight % and preferably about 0.05 weight % to about 0.3 weight %.

Embodiments of the present disclosure also include optionally adding to the above photocatalytic agents and optionally along with an oxidizing agent, at least one pH adjuster, preferably an oral or pharmaceutically acceptable pH adjuster, Such pH adjusters that may be employed include, for example, sodium bicarbonate, sodium hydroxide, potassium hydroxide, triethanolamine (an organic compound composed of a tri-alcohol & an amine), or a combination thereof. For the aspects of the disclosed embodiments, the pH adjuster, (e.g., sodium bicarbonate) can be employed at concentrations of from about 5 weight % to about 99.5 weight %, preferably from about 10 weight % to about 80 weight %, except when it is in powder form where it is preferably from about 90 weight % to about 99.5 weight %. For a combination of sodium bicarbonate and sodium hydroxide, sodium bicarbonate can be employed at concentrations preferably from about 15 weight % to about 79 weight % and sodium hydroxide can be employed at concentrations preferably from about 1 weight % to about 5 weight %. The teeth whitener pre-treatment composition embodiment including a phtotocatalytic agent and pH adjuster should be at a pH of above about 7.5, preferably above a pH of about 8, more preferably above a pH of about 9 with a maximum pH of less than about 10. These embodiments can be combined with the device embodiments of the present disclosure and the use thereof, such that the device is used after the photocatalytic agent and pH adjuster and/or oxidizing agent (separately, together or photocatalytic agent and pH adjuster followed by oxidizing agent) are applied to the teeth.

One embodiment that includes the teeth whitener pre-treatment composition including photocatalytic agent with or without a pH adjuster is administered to the teeth followed by the administration of the teeth whitener composition (i.e., oxidizing or bleaching agent). This treatment regimen can range from about 1 20 min session to about 2 to about 6 20 min sessions completed over a span of a several days to a week. For example, where the teeth whitener pre-treatment composition including photocatalytic agent and pH adjuster is administered to the teeth prior to the administration of the teeth whitener composition (i.e., oxidizing or bleaching agent) and the teeth whitener composition includes a hydrogen peroxide base concentration of about 14 weight %, the treatment regimen can range from about 1 20minute session to about 6 20 min sessions completed over a span of a several days to a week.

One embodiment of the present disclosure is a teeth whitener pre-treatment composition includes a pH adjuster, for example, sodium bicarbonate at a concentration of from about 5 weight % to about 99.5 weight %, preferably from about 40 weight % to about 80 weight %, and a photocatalytic agent, for example, magnesium gluconate at a concentration of from about 0.05 weight % to about 20 weight %, preferably from about 0.15 weight % to about 5 weight %, more preferably from about 0.15 weight % to about 1 weight %.

Peroxide and other teeth whitening agents are known to have a whitening effect on the teeth by applying them to a tooth surface in various forms, such as, for example, tray-based gels, peroxide strips and paint on whiteners. By applying a teeth whitener pre-treatment composition including, for example, a mixture of a photocatalytic agent (e.g., a metal salt catalyst) and a pH adjuster or a pH adjuster alone to the tooth surface prior to applying a teeth whitening oxidizing composition, the operation and effect of the teeth whitening composition can be improved (e.g., accelerated). Such improvement may be attributable to the tooth whitener pre-treatment composition speeding up the breakdown of the teeth whitener compositions (i.e. for a peroxide base teeth whitener composition, the breakdown of peroxide into oxygen radicals to oxidize the stains in the tooth surface). The pH adjuster may raise the pH to a desired level and a metal salt catalyst may catalyze the peroxide breakdown. The pH adjuster should raise the pH of the mixture above about 7.5, preferably above a pH of about 8, more preferably above a pH of about 9 with a maximum pH of less than about 10.8.

The teeth whitener pre-treatment composition can include separate application of the photocatalytic agent (e.g., metal salt catalyst) and a pH adjuster to the tooth surface or application of a mixture of photocatalytic agent (e.g., metal salt catalyst) and a pH adjuster to the tooth surface or a pH adjuster alone to the tooth surface. Where the photocatalytic agent (e.g., metal salt catalyst) and a pH adjuster are separately applied to the tooth surface, either can be applied first. Such application can be followed by application of an oxidizing or bleaching tooth whitening agent (e.g., one of the peroxides).

The mixture or separate photocatalytic agent (e.g., metal salt catalyst) and a pH adjuster or pH adjuster alone can be administered, for example, in a paste form, gel form, a liquid form, a rinse form or spray form. For these forms for administration or application to the teeth that are not in substantially dry powder form, the mixture or separate photocatalytic agent (e.g., metal salt catalyst) and a pH adjuster or a pH adjuster alone can be also include vehicles, such as, for example, glycerin, propylene glycol, polyvinylpyrrolidone (PVP), distilled water or combinations thereof. If the mixture or separate metal salt catalyst and a pH adjuster or pH adjuster alone are administered in powder form, excipients or other vehicle components are not necessary.

Another embodiment of the disclosure relates to compositions and methods of using same including the use of a hydrating agent including, for example, mineral oil, glycerin, and coconut oil which may be used alone or with a oxidizing or bleaching agent such as carbamide peroxide, hydrogen peroxide, calcium peroxide, zinc peroxide, sodium chlorite, and sodium perborate but preferably hydrogen peroxide. The hydrating agent with or without oxidizing agent may be formulated, for example as a paste form, gel form, a liquid, spray, or paint that can be applied to the tooth labial surface. Where the embodiment includes an oxidizing agent, the concentration of oxidizing agent can be in the range of from about 0.01 to about 5 percent by weight, preferably about 0.01 to about 3 percent by weight or about 0.1 percent by weight. The present disclosure may comprise a variety of different oxidizing agents, peroxide types, and concentrations. The preferred embodiment is 0.1% H₂O₂ in the hydrating agent, e.g., mineral oil. These embodiments can be combined with the device embodiments of the present disclosure and the use thereof, such that the device is used after the hydrating agent with or without oxidizing agent is applied to the teeth.

Other embodiments of the present disclosure include a method of applying at least one of the teeth whitener pre-treatment compositions disclosed herein to a tooth, teeth or other oral surfaces. Still other embodiments include a method of applying at least one of the teeth whitener pre-treatment compositions of the present disclosure to a tooth, teeth or other oral surfaces prior to applying a teeth whitener composition (e.g., including an oxidizing or bleaching agent alone or in combination with other ingredients included in embodiments of the present disclosure), as part of a teeth whitening treatment regimen (a teeth whitening treatment regimen including applying a teeth whitener composition to the tooth, teeth and other oral surfaces). Still other embodiments include a method of applying at least one of the teeth whitener pre-treatment compositions of the present disclosure to a tooth, teeth or other oral surfaces followed by a teeth whitening treatment regimen. Still more embodiments include a method of applying at least one of the teeth whitener pre-treatment compositions of the present disclosure to a tooth, teeth or other oral surfaces followed by applying a teeth whitener composition to the same a tooth, teeth or other oral surfaces to which was applied the at least one of the teeth whitener pre-treatment compositions of the present disclosure.

Other embodiments of the present disclosure include a method of accelerated breakdown of peroxide into oxygen radicals by applying at least one of the teeth whitener pre-treatment compositions of the present disclosure to a tooth, teeth or other oral surfaces followed by applying a teeth whitener composition including peroxide to the same to a tooth, teeth or other oral surfaces. Still other embodiments include a method of raise the pH to a desired level (above about 6.5, preferably above a pH of about 8, more preferably above a pH of about 9 with a maximum pH of less than about 10.8) and catalyzing the peroxide breakdown of a teeth whitener composition including peroxide by applying at least one of the teeth whitener pre-treatment compositions of the present disclosure to a tooth, teeth or other oral surfaces followed by applying a teeth whitener composition including peroxide. Still other embodiments include a method of priming a tooth or teeth surface for a teeth whitening treatment regimen by applying at least one of the teeth whitener pre-treatment compositions of the present disclosure to a tooth, teeth or other oral surfaces prior to applying a teeth whitener composition.

Still other embodiments include a method of applying at least one of the teeth whitener pre-treatment compositions of the present disclosure to a tooth, teeth or other oral surfaces, followed by applying a teeth whitener composition including peroxide to the same tooth, teeth or other oral surfaces and followed by irradiating the same tooth, teeth or other oral surfaces with blue, violet or indigo light.

Still other embodiments include a method of whitening a tooth or teeth surface using a teeth whitening treatment regimen by applying at least one of the teeth whitener pre-treatment compositions of the present disclosure to a tooth, teeth or other oral surface prior to applying a teeth whitener composition and applying a teeth whitener composition as part of a teeth whitening treatment regimen.

Still other embodiments include all of the method embodiments of the present disclosure combined with the device embodiments of the present disclosure and the use thereof.

The oral care compositions of the present disclosure can include oral rinses (e.g. mouthrinses or washes), pastes, dentifrices, and oral gels. Oral care compositions include products which, in the ordinary course of usage, are not intentionally swallowed for purposes of systemic administration of particular therapeutic agents, but is rather retained in the oral cavity or the tissues thereof for a time sufficient to be effective for purposes of therapeutic activity within the oral cavity and surfaces and tissues therein, including but not limited to teeth whitening. teeth, as used herein, refers to natural teeth as well as artificial teeth or dental prosthesis. Oral cavity includes teeth, tissues (including mucous membranes and cheek tissue in the oral cavity) and the surfaces thereof present in mouth.

Another embodiment is a kit that includes an embodiment of at least one of the embodiments of the present disclosure, at least one of the device embodiments of the present disclosure and may also include instructions for use. Another embodiment is a kit that includes at least one of the teeth whitener pre-treatment compositions of the present disclosure and a teeth whitener composition, at least one of the device embodiments of the present disclosure and may also include instructions for use.

Other optional components of the embodiments of the present disclosure can include one or more of the following: a preservative, a binder, absorbents (bulking agents), a humectant, a flavoring agent, a chelating agent, a sweetener, an occlusive agent (to prevent or retard water loss of a composition to which it is part), an anti-bad-breath agent, a viscosity modifier or thickener, an abrasive, a fluoride source, an agent used to diminish teeth sensitivity, a remineralizing agent, and a skin or oral tissue-conditioning agents.

Embodiments of the present disclosure can also include sources of fluoride ions, or fluorine-providing compounds useful, for example, as anti-caries agents known for their ability to release fluoride ions in water. An oral or pharmaceutically acceptable fluoride source can include one or a mixture of fluoride source, such as, for example, fluoride salts, such as soluble alkali metal and alkaline earth metal salts, for example, sodium fluoride, potassium fluoride, ammonium fluoride, calcium fluoride, copper fluorides such as cuprous fluoride, zinc fluoride, barium fluoride, sodium fluorosilicate, ammonium fluorosilicate, sodium fluorozirconate, sodium monofluoro-phosphate, aluminum mono- and di-fluorophosphate, and fluorinated sodium calcium pyrophosphate. Alkali metal and tin fluorides, such as sodium and stannous fluoride, sodium monofluorophosphate, and mixtures thereof, are preferred. It is preferred to have the fluoride source in the first oral care composition at concentrations ranging from about 0.005 weight % to about 3 weight %.

An oral or pharmaceutically acceptable abrasive or polishing agent can include one or a mixture of abrasives such as for example, calcium carbonate, sodium metaphosphate, potassium metaphosphate, tricalcium phosphate, dihydrated dicalcium phosphate, calcined alumina and siliceous materials, bentonite clay, kaolin clay or combinations thereof. Preferred abrasives include calcium carbonate, siliceous materials, such as silica and more preferably hydrated silica, bentonite clay, kaolin clay or combinations thereof. More preferably abrasives include a mixture of calcium carbonate, bentonite clay, kaolin clay and hydrated silica. The abrasive content the first oral care composition embodiments can be in the range of from about of 1 weight % to about 80 weight % and preferably about 23 weight % to about 50 weight %.

An oral or pharmaceutically acceptable anti-bad-breath agent which are suitable for treating bad breath (e.g. halitosis). can include one or a mixture of anti-bad-breath agents such as for example, cetylpyridinium chloride (CPC), zinc compounds (e.g., zinc chloride, zinc citrate and Zinc gluconate), and chlorhexidine, preferably Zinc Chloride. The anti-bad-breath agents content the first oral care composition embodiments can be in the range of from about of 0.05 weight % to about 5 weight % and preferably about 0.075 weight % to about 2 weight %.

An oral or pharmaceutically acceptable thickener can include one or a mixture of organic and inorganic thickeners or viscosity modifiers. Organic thickeners such as natural and synthetic gums and colloids may also be incorporated in the present invention. Examples of such organic thickeners include carrageenan (Irish moss), xanthan gum and sodium carboxymethyl cellulose, starch, polyvinylpyrrolidone, hydroxyethylpropyl cellulose, hydroxybutyl methyl cellulose, hydroxypropylmethyl cellulose, and hydroxyethyl cellulose. Inorganic thickeners such as Laponite D are preferred, as well as amorphous silica compounds which function as thickening agents including, colloidal silica compounds available under tradenames such as Cab-o-sil fumed silica manufactured by Cabot Corporation and distributed by Lenape Chemical, Bound Brook, N.J., Zeodent 165 from J. M. Huber Chemicals Division, Havre de Grace, Md. 21078 and Sylox 15 from Grace Davison, Baltimore, Md. 21203.

An oral or pharmaceutically acceptable binder, that can also act a viscosity increasing agent, can include one or a mixture of binders, such as for example, water soluble binders, such as, for example polyvinyl alcohols, polyvinyl pyrrolidone (PVP), polyvinyl pyrrolidone derivates and copolymers, saccharides such as D-glucose, D-fructose, sorbitol, polysaccharides such as starch, cellulose and pectin including derivatives thereof, gelatin, naturally occurring substances such as alginic acid, carrageen gum, locust bean gum, guar gum, xanthan gum, tragacanth gum, arabic gum, karaya gum. Preferably, for the first oral care composition, polyvinyl pyrrolidone (PVP) as well as cellulose gum or xanthan gum can be used as a binder and/or thickener. The binder/thickener content the first oral care composition embodiments may be present in the first oral care composition embodiments in concentrations of about 0.1 weight % to about 10 weight %, preferably about 5 weight % to about 8 weight %.

An oral or pharmaceutically acceptable chelating agent can include one or a mixture of chelating agents, such as for example, sodium tripolyphosphate, ethylenediamine tetracetic acid (“EDTA”) and its salts (e.g., tetrasodium EDTA and calcium EDTA), or a combination thereof. For the aspects of the disclosed embodiments, such chelating agents can be employed at concentrations of from about 0.3 weight % to about 30 weight %, from about 0.1 wt % to about 10 wt %, preferably about 0.1 wt % to about 4.5 wt %. The preferred chelating agent for the first oral care composition embodiments is sodium tripolyphosphate.

An oral or pharmaceutically acceptable occlusive agent can include one or a mixture of occlusive agents, such as, for example, petrolatum, mineral oil, Wax includes synthetic and natural waxes (such as paraffins, beeswax, sugarcane wax, retamo wax, lanolin, petroleum jelly, vegetable waxes (including Bayberry wax, candelilla wax, carnauba wax, Castor wax, Esparto wax, Japan wax, Jojoba oil, Rice bran wax or Soy wax), silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil. Synthetic fatty acid esters such as cetyl palmitate and are additional examples of useful waxes. Such occlusive agents may have other formulation benefits as well. The occlusive agent content the first oral care composition embodiments can be in the range of from about of 1 weight % to about 35 weight % and preferably about 1 weight % to about 10 weight %. coconut oil is the preferred occlusive agent for the first oral composition.

An oral or pharmaceutically acceptable humectant can include one or a mixture of humectants, such as, for example, glycerin, sorbitol and polyethylene glycol, preferably, a mixture of glycerin and sorbitol for the first oral care composition embodiments and preferably propylene glycol and glycerin for the second oral care composition embodiments. The humectant content the first oral care composition embodiments and the second oral second composition embodiments can be in the range of from about of 1 weight % to about 30 weight % and preferably about 2 weight % to about 25 weight %.

An oral or pharmaceutically acceptable flavoring agent can include one or a mixture of flavoring agents, such as for example bubble gum flavor, cherry flavor, grape flavor, anise oil, cassia oil, vanilla extract, vanilla creme, orange flavor, anethole, licorice, spearmint oil (e.g., spearmint native or essential oil), peppermint oil, phenylacetaldehyde diisobutyl acetal, and mixtures thereof, preferably spearmint essential oil. Some flavoring agents can also act as sweeteners and can be use as such and include, for example, neohespiridin dehydrochalcone, xylitol, Sucralose, and mixtures thereof, preferably xylitol. The flavoring agent content the first oral care composition embodiments and the second oral care composition embodiments can be in the range of from about of 0.05 weight % to about 3 weight % and preferably about 0.5 weight % to about 1 weight %.

An oral or pharmaceutically acceptable agent used to diminish teeth sensitivity in either the first or second oral care composition embodiments can include one or a mixture of such agents, such as, for example, strontium chloride, potassium nitrate and potassium citrate. The agent used to diminish teeth sensitivity content can be in the range of from about of 0.1 weight % to about 5 weight %. A preferred such agent in the second oral care composition embodiments is potassium nitrate.

Calcium glycerophosphate and nano HAP (hydroxyapatite) are also a dental agent is capable of remineralizing enamel and can be optionally included in the embodiments of the present disclosure and applied after the tooth whitening regime is completed using the methods and composition embodiments of the present disclosure with the device embodiments of the present disclosure. In certain embodiments of the present disclosure, such a remineralizing agent can also include a phosphate compound, a calcium compound, a calcium phosphate compound, hydroxyapatite and a caseinate. The phosphate compound can include, for example, a monobasic phosphate compound, a dibasic phosphate compound, a tribasic phosphate compound, calcium glycerophosphate, and combinations thereof.

Preservatives can include, for example, potassium sorbate, sodium benzoate, grapefruit seed extract and mixtures thereof. Preservatives can be generally used at levels of from about 0.5 wt % to about 2 wt %, by weight of the composition.

Absorbents (bulking agents) can include, for example, bentonite, hydrated silica, kaolin clay, and mixtures thereof. Absorbents (bulking agents)can be generally used at levels of from about 1 wt % to about 10 wt %, about 1 wt % to about 4.5 wt %, preferably about 1.45 wt %, by weight of the composition.

Skin or oral tissue-conditioning agents can include, for example, glycerin, hydrated silica, xylitol, xanthan gum, aloe vera and mixtures thereof. Skin-conditioning agents can be generally used at levels of from about 2.5 wt % to about 7 wt %, by weight of the composition.

The present disclosure may also include the use of an activation indicator which can change color upon initiation or completion. This may include methylene blue, yellow compliment color purple or other such agents known to those skilled in the art, including any mixture thereof.

The present disclosure may also include post treatment applications including nanoparticle pore sealants, remineralizers, germ fighting compounds, breath fresheners, flavorings and sweeteners, or other such agents known to those skilled in the art, including any mixture thereof.

Another embodiment of the disclosure relates to a method of whitening kit for home use of a whitening agent comprising a composition of zinc oxide, a composition of hydrogen peroxide and a light source.

Another embodiment of the disclosure relates to the application of the bleaching formulation wherein the treatment period is from about 8-about 25 minutes, more preferably about 15-about 20 minutes, more preferably about 20 minutes or less. Repeat treatment is also preferred with two to 6 sessions being common. More preferably 3-4 sessions (5 to 6being most common) are sufficient for achieving desired whiteness. Sessions are typically divided by 24 hrs (six to twelve hours also being very common) but can be longer or shorter depending on individual conditions. Total bleaching time is between about 20 to about 80 minutes, more preferably about 40 minutes (wherein each session is approximately 20 minutes). These embodiments can be combined with the device embodiments of the present disclosure and the use thereof, such that the device is used after the above components are applied to the teeth as disclosed herein.

Another embodiment includes compositions and methods of the present disclosure include embodiments having oral care compositions that are a pre-treatment of the tooth surface to accelerate or catalyze teeth whitener compositions prior to application of the whitening/bleaching agent , the latter with or without a photocatalytic agent, that can be used to oxidize stains (e.g. organic stains) on the teeth, for example, those teeth whitener compositions that include a peroxide base tooth whitening agent. These embodiments can be combined with the device embodiments of the present disclosure and the use thereof, such that the device is used after the above components are applied to the teeth as disclosed herein.

Peroxide and other teeth whitening agents are known to have a whitening effect on the teeth by applying them to a tooth surface in various forms, such as, for example, tray-based gels, peroxide strips and paint on whiteners. By applying a teeth whitener pre-treatment composition including a mixture of a metal salt catalyst and a pH adjuster to the tooth surface prior to applying the teeth whitener compositions, the operation and effect of the teeth whitener composition can be improved (e.g., accelerated). Such improvement may be attributable to the tooth whitener pre-treatment composition speeding up the breakdown of the teeth whitener compositions (i.e. for a peroxide base teeth whitener composition, the breakdown of peroxide into oxygen radicals to oxidize the stains in the tooth surface). The pH adjuster may raise the pH to a desired level and a metal salt catalyst may catalyze the peroxide breakdown.

The teeth whitener pre-treatment composition can include separate application of the metal salt catalyst and a pH adjuster to the tooth surface or application of a mixture of the metal salt catalyst and a pH adjuster to the tooth surface. Where the metal salt catalyst and a pH adjuster are separately applied to the tooth surface, either can be applied first. These embodiments can be combined with the device embodiments of the present disclosure and the use thereof, such that the device is used after the above components are applied to the teeth as disclosed herein.

The teeth whitener compositions can be applied to the tooth surface after the teeth whitener pre-treatment composition is applied to the tooth surface. The teeth whitener compositions can include peroxide teeth whitener compositions and can be, for example, hydrogen peroxide, carbamide peroxide, calcium peroxide, sodium perborate, sodium percarbonate, sodium chlorite, or any mixture thereof. that is placed on the tooth in the second step. The delivery of the teeth whitener compositions in the second step after the teeth whitener pre-treatment composition is applied can be in gel in tray form, adhesive strips form, gel painted on form or gel placed on a toothbrush and brushed with. These embodiments can be combined with the device embodiments of the present disclosure and the use thereof, such that the device is used after the above components are applied to the teeth as disclosed herein.

For embodiments including a teeth whitener pre-treatment composition (including e.g., photocatalytic agent, pH adjuster, etc.) that is administered to the teeth prior to the administration of the teeth whitener composition (oxidizing agent), the pre-treatment can be administered for a period of time ranging from about 30 seconds to about 2 minutes, preferably about 1 minute prior to administering the teeth whitener composition (oxidizing agent) which is followed by the device embodiments of the present disclosure and the use thereof,. When the teeth whitener pre-treatment composition is administered to the teeth prior to the administration of the teeth whitener composition, for a hydrogen peroxide base teeth whitener composition, hydrogen peroxide can be employed at concentrations of from about 3 weight % to about 30 weight %, preferably from about 12 weight % to about 14 weight %.

Where the teeth whitener pre-treatment composition is administered to the teeth prior to the administration of the teeth whitener composition and the teeth whitener composition includes a hydrogen peroxide base concentration of about 14 weight %, the treatment regimen can range from about 1 20-minute session to about 6 twenty min sessions completed over a span of a several days to a week.

One embodiment of the present disclosure is a teeth whitener pre-treatment composition and use thereof including a pH adjuster, for example, sodium bicarbonate at a concentration of from about 5 weight % to about 99.5 weight %, preferably from about 70 weight % to about 80 weight %, and a photocatalytic agent, for example, magnesium gluconate at a concentration of from about 0.05 weight % to about 20 weight %, preferably from about 0.5 weight % to about 1 weight %. These embodiments can be combined with the device embodiments of the present disclosure and the use thereof, such that the device is used after the above components are applied to the teeth as disclosed herein.

Other embodiments of the present disclosure include a method of applying at least one of the teeth whitener pre-treatment compositions disclosed herein to a tooth, teeth or other oral surfaces. Still other embodiments include a method of applying at least one of the teeth whitener pre-treatment compositions of the present disclosure to a tooth, teeth or other oral surfaces prior to applying a teeth whitener composition, as part of a teeth whitening treatment regimen (a teeth whitening treatment regimen including applying a teeth whitener composition to the tooth, teeth and other oral surfaces). Still other embodiments include a method of applying at least one of the teeth whitener pre-treatment compositions of the present disclosure to a tooth, teeth or other oral surfaces followed by a teeth whitening treatment regimen. Still more embodiments include a method of applying at least one of the teeth whitener pre-treatment compositions of the present disclosure to a tooth, teeth or other oral surfaces followed by applying a teeth whitener composition to the same a tooth, teeth or other oral surfaces to which was applied the at least one of the teeth whitener pre-treatment compositions of the present disclosure. These embodiments can be combined with the device embodiments of the present disclosure and the use thereof, such that the device is used after the above components are applied to the teeth as disclosed herein.

Other embodiments of the present disclosure include a method of accelerated breakdown of peroxide into oxygen radicals by applying at least one of the teeth whitener pre-treatment compositions of the present disclosure to a tooth, teeth or other oral surfaces followed by applying a teeth whitener composition including peroxide to the same to a tooth, teeth or other oral surfaces. Still other embodiments include a method of raise the pH to a desired level and catalyzing the peroxide breakdown a teeth whitener composition including peroxide by applying at least one of the teeth whitener pre-treatment compositions of the present disclosure to a tooth, teeth or other oral surfaces followed by applying a teeth whitener composition including peroxide. Still other embodiments include a method of priming a tooth or teeth surface for a teeth whitening treatment regimen by applying at least one of the teeth whitener pre-treatment compositions of the present disclosure to a tooth, teeth or other oral surfaces prior to applying a teeth whitener composition. These embodiments can be combined with the device embodiments of the present disclosure and the use thereof, such that the device is used after the above components are applied to the teeth as disclosed herein.

Still other embodiments include a method of applying at least one of the teeth whitener pre-treatment compositions of the present disclosure to a tooth, teeth or other oral surfaces, followed by applying a teeth whitener composition including peroxide to the same tooth, teeth or other oral surfaces and followed by irradiating the same tooth, teeth or other oral surfaces with light, for example, violet/indigo light, preferably, violet light. These embodiments can be combined with the device embodiments of the present disclosure and the use thereof, such that the device is used after the above components are applied to the teeth as disclosed herein.

Still other embodiments include a method of whitening a tooth or teeth surface using a teeth whitening treatment regimen by applying at least one of the teeth whitener pre-treatment compositions of the present disclosure to a tooth, teeth or other oral surface prior to applying a teeth whitener composition and applying a teeth whitener composition as part of a teeth whitening treatment regimen. These embodiments can be combined with the device embodiments of the present disclosure and the use thereof, such that the device is used after the above components are applied to the teeth as disclosed herein.

Another embodiment is a kit that includes an embodiment of at least one of the teeth whitener pre-treatment compositions of the present disclosure and may also include instructions for use. Another embodiment is a kit that includes at least one of the teeth whitener pre-treatment compositions of the present disclosure and a teeth whitener composition and may also include instructions for use. These embodiments can be combined with the device embodiments of the present disclosure and the use thereof, such that the device is used after the above components are applied to the teeth as disclosed herein.

The following examples demonstrate methods or uses to successfully whiten a patient's teeth.

### EXAMPLE 1

1. Zinc oxide is painted on teeth labial surface in a gel carrier form with the zinc oxide (USP grade) in a concentration of about 1 to about 6% with the size of the particle ranging from about 400 to about 420 nm, preferably about 405 nm to about 410 nm.

2. Hydrogen peroxide gel in a concentration of about 9% (can be used at about 12% or about 14%) is painted on the labial surface of the teeth creating an admixture of the zinc oxide and peroxide at the tooth surface (steps 1 and 2 are interchangeable).

3. An illuminator LED light source array coupled with a dual cylindrical lens mouthguard pulsating at a frequency of about 33 Hertz with a 5% duty cycle (5% ON time) and a wavelength of about 405 to 410 nm is directed at the tooth surface and away from the oral tissues.

4. After treatment period (e.g., 5, 7, 30 or 35 minutes) the light source is removed. This procedure may be repeated 3-6 times (such as daily or twice daily) with light source removed after each treatment (e.g., 15, 20 or 30 minutes).

### EXAMPLE 2

The zinc and peroxide components are maintained in two separate chambers of a pen or syringe and mix at the delivery tip as the components are applied to the tooth. Once the zinc and peroxide components have been applied to the teeth an LED violet/indigo (preferably violet) light source is directed at the tooth surface and away from the oral tissues.

### EXAMPLE 3

The zinc and peroxide components are maintained in two separate chambers of a pen or syringe or two separate pens or syringes. Zinc oxide (USP grade) is painted on teeth labial surface in a gel carrier form at a concentration of about 1% to about 6% with the size of the particle ranging from about 0.5 to 10 microns. Hydrogen peroxide gel in a concentration of about 9% to about 14% is painted on the labial surface of the teeth on top of the zinc oxide creating an admixture of the zinc oxide and peroxide at the tooth surface. The teeth are then illuminated as described above in Example 1.

### EXAMPLE 4

Prepaste ingredients Sodium Bicarbonate, Glycerin, Water, Xylitol, Bentonite, Zinc Oxide, Xanthan Gum, Sucralose, PVP, Potassium Sorbate, Sodium Benzoate, Mentha Viridis (Spearmint) Leaf Oil, Disodium EDTA, Silica, Magnesium Gluconate.

### EXAMPLE 5

Water (solvent), Hydrogen Peroxide (oxidizing agent), Xylitol (sweetener), Glycerin (humectant), Carbomer (emulsifier), PEG-60 Hydrogenated Castor Oil (surfactant), Ammonium Hydroxide (pH adjuster), Flavor, Etidronic Acid (bleaching agent or peroxide stabilizer), Potassium Stannate (bleaching agent or peroxide stabilizer).

### EXAMPLE 6

Propylene Glycol (humectant—about 20%- about 40%); (purified) water (solvent—about 20 weight % to about 40 weight %), Glycerin (humectant—about 20 weight % to about 40 weight %), Hydrogen Peroxide (oxidizing agent—about 14 weight %), triethanolamine (pH adjuster—about 1 weight % to about 10 weight %), Carbomer (emulsifier—about 1 weight % to about 10 weight %), Xylitol (sweetener—about 1 weight %), Spearmint Essential Oil (flavor—less than about 1 weight %), Potassium Nitrate (sensitivity reducer—less than about 1 weight %)

### EXAMPLE 7

### Teeth Whitener Pre-Treatment (Pre-Paste)

Ingredients: sodium bicarbonate granular (abrasive; skin protectant) 48.5 wt %; glycerin (skin-conditioning agent) 32.97 wt %; DI water (skin-conditioning agent) 9 wt %; hydrated silica (skin-conditioning agent) 3 wt %; xylitol (skin-conditioning agent) 1.5 wt %; bentonite (absorbent; bulking agent) 1.45 wt %; sucralose (flavoring agent) 0.85 wt %; potassium sorbate (preservative) 0.62 wt %; sodium benzoate (preservative) 0.6 wt %; xanthan gum (skin-conditioning agent) 0.59 wt %; PVP K-30 (binder) 0.35 wt %; disodium EDTA (chelating agent) 0.22 wt %; magnesium gluconate (skin-conditioning agent) 0.2 wt %; and spearmint native oil (flavoring agent) 0.15 wt %. Each of the ingredients may have additional functions along with the stated functions.

### EXAMPLE 8

### Teeth Whitener Pre-Treatment With Zinc for Use With Light as Part of the Whitening Process

Ingredients: sodium bicarbonate granular (abrasive; skin protectant) 48.5 wt %; glycerin (skin-conditioning agent) 31.97 wt %; zinc oxide (photocatalytic agent) 1 wt %; DI water (skin-conditioning agent) 9 wt %; hydrated silica (skin-conditioning agent) 3 wt %; xylitol (skin-conditioning agent) 1.5 wt %; bentonite (absorbent; bulking agent) 1.45 wt %; sucralose (flavoring agent) 0.85 wt %; potassium sorbate (preservative) 0.62 wt %; sodium benzoate (preservative) 0.6 wt %; xanthan gum (skin-conditioning agent) 0.59 wt %; PVP K-30 (binder) 0.35 wt %; disodium EDTA (chelating agent) 0.22 wt %; magnesium gluconate (skin-conditioning agent) 0.2 wt %; and spearmint native oil (flavoring agent) 0.15 wt %. Each of the ingredients may have additional functions along with the stated functions.

### EXAMPLE 9

### Laboratory Investigation of the Whitening Effect on Extrinsic Dental Stain Using a Sponsored Supplied Brush-On Paste/Whitening Pen Gel Combo & a Novel Vibrating Intraoral Led Light

PURPOSE: The purpose of this 2-cell laboratory study was to evaluate the whitening effect on extrinsically stained bovine enamel of a sponsored supplied combo brush-on paste/whitening pen and vibrating LED light at different time periods.

Procedure

Specimen Preparation: Approximately 4-mm squares of dental enamel were cut, using a diamond cutting disk, from bovine permanent incisors. Using a mold, the enamel squares are embedded in clear orthodontic repair resin (Lang Jet Powder, Lang Dental Mftg, Wheeling Ill. 60090-0969) to provide 1.5 cm square blocks with the labial surface exposed.

Stain Measurement: For balancing purposes, the intensity of the intrinsic stain within the teeth were measured instrumentally, both before and after treatment by taking diffuse reflectance absorbance readings with a specially-designed Minolta spectrophotometer (Minolta CM-503i Spectrophotometer with diffuse illumination/8 viewing angle and 3 mm aperture, Minolta Camera Co., 101 Williams Drive, Ramsey, N.J., 07446). Absorbance measurements over the entire visible color spectrum are obtained using the CIELAB color scale (Commission Internationale de L'Eclairage. Recommendations on uniform color spaces.

Color difference equations. Psychometric color terms. Suppl 2 to CIE publication 15 (E-13.1) 1971/(TC-1.3), 1978, Paris: Bureau Central de la CIE, 1978). There were 6 specimens per cell.

Measurements were conducted by aligning the center of the 4-mm circular segment of exposed enamel directly over the 3-mm diameter aperture of the Minolta spectrophotometer using the instrument's target mask, which enables accurate positioning of the specimens each time. The intrinsic color of the teeth are recorded in triplicate using the tristimulus L*a*b* color space as established by the Commission Internationale de L'Eclairage (CIE). The changes in tooth color are compared using the color difference equation ΔE=[(ΔL*)²+(Δa*)²+(Δb*)²]. The symbols ΔL*, Δa*, and Δb* represent the changes in white-black, red-green, and yellow-blue, respectively. The greater the value for the calculated ΔE, the greater is the increased whiteness of the teeth. The individual color factors are analyzed separately to determine the color range most affected by the oral devices.

Test Products:

Sponsor supplied pre-paste (non-descript blank tube)

Intelliwhite 14% H2O2 whitening pen

LED vibrating intraoral light

Treatment

1. A half a pea sized ribbon of pre-paste was placed on a dry stained enamel surface

2. Brushed tooth surface with an Oral-B standard Soft toothbrush (DRY) for 30 sec.

3. Did not rinse

4. Painted a thin layer of whitening gel on dry tooth surface and left on for 10 min

5. Placed stain specimen against the front of the intraoral whitening light (attached with rubber bands)

6. Placed LED light and stain specimen in a small box and closed the lid for the duration of treatment.

7. Turned the light on and vibration motor turned on for 15 sec, then shut itself off. The full light spectrum stayed on for an additional 15 min.

8. After 15 min (Gp 1) and 20 min (Gp 2), the specimens were rinsed and wiped off and placed in artificial saliva. After all specimens were treated, spectrophotometer readings took place.

Repeated Steps 1-8 for the 2^{nd} treatment cycle

Group 1 had readings at 15 min and 30 min

Group 2 had readings at 20 min and 40 min

DATA ANALYSIS: The difference between the pre-test and post-test readings for each color factor (ΔL*, Δa*, and Δb*) represents the ability of the whitening devices to remove intrinsic tooth stain by whitening. The overall change in stain is calculated using the CIELAB equation ΔE=[(ΔL*)²+(Δa*)²+(Δb*)²]. Each component of the L*a*b* scale also is analyzed separately to determine the specific changes in white-black, red-green, and yellow-blue, respectively.

Summary

| | | Baseline | | | 15 min | | | 15 min difference | | | |
|---|---|---|---|---|---|---|---|---|---|---|---|
| ID | Gp | L* | a* | b* | L* | a* | b* | L* | a* | b* | E |
| A | 1 | 33.66 | 10.49 | 13.14 | 49.89 | 8.84 | 26.81 | 16.23 | 1.65 | 13.67 | 21.41 |
| | | Baseline | | | 20 min | | | 20 min difference | | | |
| ID | Gp | L* | a* | b* | L* | a* | b* | L* | a* | b* | E |
| B | 2 | 33.43 | 10.76 | 12.67 | 55.91 | 6.86 | 21.61 | 22.49 | 3.90 | 8.94 | 24.90 |

| | | 30 min | | | 30 min difference | | | |
|---|---|---|---|---|---|---|---|---|
| ID | Gp | L* | a* | b* | L* | a* | b* | E |
| A | 1 | 59.30 | 5.48 | 11.01 | 25.64 | 5.01 | 2.13 | 26.30 |
| | | 40 min | | | 40 min difference | | | |
| ID | Gp | L* | a* | b* | L* | a* | b* | E |
| B | 2 | 67.60 | 1.93 | 7.10 | 34.17 | 8.83 | 5.56 | 35.79 |

DISCUSSION: After only the 1st treatment, group 2 (20 min) was significant from group 1 (15 min) when looking at ΔL*, Δb* and E color parameters. After the 2^{nd} treatment, all parameters from group 2 (40 min treatment time) ΔL*, Δa*, Δb* and E separated itself apart from group 1 (30 min total treatment time).

CONCLUSION: Both groups exhibited obvious whitening, with each allotted time point of exposure. This in vitro model shows that with extended treatment time, you can reasonably expect further whitening using the pre-paste, gel pen and LED light combo.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental novel features of the disclosure as applied to the exemplary embodiments thereof, it will be understood that various omissions and substitutions and changes in the form and details of devices and methods illustrated, and in their operation, may be made by those skilled in the art without departing from the spirit or scope of the disclosure. Moreover, it is expressly intended that all combinations of those elements and/or method steps, which perform substantially the same function in substantially the same way to achieve the same results, are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. It is the intention, therefore, to be limited only as indicated by the scope of the claims appended hereto.

1. An illuminating device for whitening the teeth in the mouth of a user comprising a light source that generates a wavelength in the violet portion of the electromagnetic spectrum in a wavelength of light of from about 400 nm to about 420 nm that is coupled to a mouthpiece.

2. The illuminating device according to claim 1, wherein the wavelength of light is in the violet portion of the electromagnetic spectrum in a wavelength of from about 405 nm to about 410 nm.

3. The illuminating device according to claim 1, wherein the wavelength of light is in the violet portion of the electromagnetic spectrum in a wavelength of about 405 nm.

4. The illuminating device according to claim 1, wherein the mouthpiece is removable from the LED light source.

5. The illuminating device according to claim 1, further including an electrical conduit having first and second ends, the first end electrically connected to the LED light source to provide electrical power thereto and the second end including a connector to electrically connect the electrical conduit to a power source.

6. The illuminating device according to claim 5, wherein the power source is an electrical outlet, a cellphone, an electronic tablet or a computer.

7. An oral treatment device for whitening the teeth in the mouth of a user, comprising:
a mouthpiece sized and shaped so that it can be inserted into the user's oral cavity and including:
an inner border having a substantially concave shape to substantially match the curvature of rows of the teeth in the mouth;
a light generating element positioned to focus the light generated toward the teeth positioned in the mouthpiece; and
a plurality of projections on the inner border of the mouthpiece positioned adjacent the teeth positioned in the mouthpiece;
a control unit connected to the mouthpiece, the control unit including:
a vibration generating device for generating vibrations to cause the mouthpiece to vibrate; and
a controller configured to:
selectively energize the vibration generating device; and
selectively energize light generating element.

8. The oral treatment device of claim 7, wherein the controller configured to selectively energize the vibration generating device and light generating device includes turning on and turning off each respectively.

9. The oral treatment device of claim 7, wherein the mouthpiece includes a barrier plate on which the teeth can bite down thereon and separating upper and lower teeth.

10. The oral treatment device of claim 7, wherein the oral treatment device is connected to a power source.

11. The oral treatment device of claim 7, wherein the control unit includes a power source.

12. The oral treatment device of claim 7, wherein the light generating element includes a plurality of LED lights.

13. The oral treatment device of claim 7, wherein the light generating element generates light in the violet portion of the electromagnetic spectrum in a wavelength of from about 400 nm to about 420 nm.

14. The oral treatment device of claim 7, wherein the light generating element generates light in the violet portion of the electromagnetic spectrum in a wavelength of about 405 nm.

15. A method of whitening the teeth in the mouth of a user using an oral treatment device, the oral treatment device comprising:
a mouthpiece sized and shaped so that it can be inserted into the user's oral cavity and including:
an inner border having a substantially concave shape to substantially match the curvature of rows of the teeth in the mouth;
a light generating element positioned to focus the light generated toward the teeth positioned in the mouthpiece; and
a plurality of projections on the inner border of the mouthpiece positioned adjacent the teeth positioned in the mouthpiece;
a control unit connected to the mouthpiece, the control unit including:
a vibration generating device for generating vibrations to cause the mouthpiece to vibrate; and
a controller configured to:
selectively energize the vibration generating device; and
selectively energize light generating element,
the method, comprising:
applying a tooth whitening composition to the teeth;
placing the teeth into the mouthpiece of the oral treatment device;
applying vibrations to the teeth by energizing the vibration generating device of the oral treatment device; and
applying light to the teeth by energizing the light generating element of the oral treatment device.

16. The method of claim 15, wherein the light generating element generates light in the violet portion of the electromagnetic spectrum in a wavelength of from about 400 nm to about 420 nm.

17. The method of claim 15, wherein the light generating element generates light in the violet portion of the electromagnetic spectrum in in a wavelength of about 405 nm.

18. The method of claim 15, wherein the frequency of the vibrations from the vibration generating device range from about 300 Hz to about 350 Hz.

19. The method of claim 15, wherein the step of applying vibrations to the teeth includes the controller turning on the vibration generating device of the oral treatment device and turning it off after a time period of from about 10 seconds to about 60 seconds, the step of applying light to the teeth includes the controller turning on the light generating element of the oral treatment device and turning it off after a time period of from about 10 minutes to about 30 minutes and the time period of the vibration generating device occurs during the time period of light generating element.

20. The method of claim 15, further including prior to the step of applying a tooth whitening composition to the teeth, applying a pre-treatment composition to the teeth including a photocatalytic agent or a photocatalytic agent and a pH adjuster.
